SEQUENCE LISTING (18 sequences)

SEQ ID NO: 1 - DNA, 24 bp, Artificial sequence
  Combination 1 forward primer
caattacctttacctgtcacaagc

SEQ ID NO: 2 - DNA, 22 bp, Artificial sequence
  Combination 1 reverse primer
aagccatttcttgcattcttcc

SEQ ID NO: 3 - DNA, 28 bp, Artificial sequence
  Combination 1 probe
  misc_feature (1)..(1): 6-FAM attachment
  misc_feature (9)..(9): ZEN attachment
agaggatatgacacatgaagaactgcca

SEQ ID NO: 4 - DNA, 22 bp, Artificial sequence
  Combination 2 forward primer
gcaacaagtctttctcgttctg

SEQ ID NO: 5 - DNA, 22 bp, Artificial sequence
  Combination 2 reverse primer
gactgggtttctagcagagaag

SEQ ID NO: 6 - DNA, 28 bp, Artificial sequence
  Combination 2 probe
  misc_feature (1)..(1): 6-FAM attachment
  misc_feature (9)..(9): ZEN attachment
cctcgataaacttctctgtgccatcagg

SEQ ID NO: 7 - DNA, 22 bp, Artificial sequence
  Combination 3 forward primer
acctcgataaacttctctgtgc

SEQ ID NO: 8 - DNA, 23 bp, Artificial sequence
  Combination 3 reverse primer
ctaagatggttgactgggtttct

SEQ ID NO: 9 - DNA, 26 bp, Artificial sequence
  Combination 3 probe
  misc_feature (1)..(1): 6-FAM attachment
  misc_feature (9)..(9): ZEN attachment
tcatagccatgtccatcttctctgct

SEQ ID NO: 10 - DNA, 19 bp, Artificial sequence
  Combination 4 forward primer
tggtaagcttcgatcagaa

SEQ ID NO: 11 - DNA, 23 bp, Artificial sequence
  Combination 4 reverse primer
cactggtaagtgatgcagtgaaa

SEQ ID NO: 12 - DNA, 23 bp, Artificial sequence
  Combination 4 probe
  misc_feature (1)..(1): 6-FAM attachment
  misc_feature (9)..(9): ZEN attachment
acatcctccggttctccttcagt

SEQ ID NO: 13 - DNA, 27 bp, Artificial sequence
  Combinaiton 5 forward sequence
ctgtatatctaaaccctggaatctctt

SEQ ID NO: 14 - DNA, 24 bp, Artificial sequence
  Combination 5 reverse primer
gttagaaacatgcaagatggtcag

SEQ ID NO: 15 - DNA, 24 bp, Artificial sequence
  Combination 5 probe
  misc_feature (1)..(1): 6-FAM attachment
  misc_feature (9)..(9): ZEN attachment
tggctgaagtcaaagggccaattg

SEQ ID NO: 16 - DNA, 24 bp, Artificial sequence
  Combination 6 forward primer
gacgtatggatacacctctttaat

SEQ ID NO: 17 - DNA, 22 bp, Artificial sequence
  Combination 6 reverse sequence
ccaaattcctcagaagagagag

SEQ ID NO: 18 - DNA, 30 bp, Artificial sequence
  Combination 6 probe
  misc_feature (1)..(1): 6-FAM attachment
  misc_feature (9)..(9): ZEN attachment
ttcccattaatatactcaattagagaacct

(19) United States
(12) Patent Application Publication
(10) Pub. No.: US 2024/0271227 A1
(43) Pub. Date: Aug. 15, 2024

(54) COMPOSITIONS AND METHODS FOR USE IN IDENTIFYING OR QUANTIFYING MICROORGANISMS

(71) Applicant: Bayer CropScience LP, St. Louis, MO (US)

(72) Inventors: Edgar VALENCIA-MORALES, St. Louis, MO (US); Varghese THOMAS, Davis, CA (US)

(21) Appl. No.: 18/290,188

(22) PCT Filed: May 13, 2022
(86) PCT No.: PCT/US2022/029179

§ 371 Date: Nov. 10, 2023

Related U.S. Application Data
(60) Provisional application No. 63/188,189, filed on May 13, 2021.

(51) Int. Cl. C12Q 1/689; C12Q 1/6895

(52) U.S. Cl. CPC C12Q 1/689; C12Q 1/6895

(57) ABSTRACT

Compositions and methods for identifying and/or quantifying one or more bacterial and/or fungal strain(s) are described. An example composition includes a biosample, and one or more combination(s) of a forward primer sequence, a reverse primer sequence, and a probe sequence. Each of the one or more combination(s) is specific for a single bacterial strain or a single fungal strain. The composition may be used to identify and/or quantify the bacterial strain or fungal strain when present, or identified, in the biosample.

### CROSS-REFERENCE TO RELATED-APPLICATION

This application claims the benefit of, and priority to, U.S. Provisional Patent Application No. 63/188,189 filed May 13, 2021, the entire disclosure of which is incorporated herein by reference.

### FIELD

The present disclosure generally relates to compositions and methods for identifying and/or quantifying one or more bacterial and/or fungal strains (broadly, microorganisms), for example, on a seed or in a seed wash or in a liquid formulation treatment for a seed.

### DESCRIPTION OF THE TEXT FILE SUBMITTED ELECTRONICALLY

The contents of the text file submitted electronically are incorporated herein by reference in their entirety, including a computer readable format copy of the Sequence Listing (filename: BCS206500WO_ST25.txt, file size 5 kilobytes, created on May 12, 2022).

### BACKGROUND

This section provides background information related to the present disclosure which is not necessarily prior art.

Plants, bacteria, and fungi coexist in a symbiotic relationship. Soil microbes produce essential elements, including nitrogen and phosphorus, and nutrients that the plant absorbs through the root system. Plants growing in soil lacking the proper microbes may not fully achieve their potential growth and yield.

Certain beneficial microbes (bacteria and fungi) can be introduced to crop plants and soil by coating the plant seeds with those microbes or their spores and/or by applying a liquid composition comprising those microbes or their spores to planted crops or to the soil where the crops are or will be planted.

There remains a need in the art for improved methods to identify and/or quantify the presence and/or amount of a certain microbe or the relative presence and/or amounts of a combination of microbes in a formulation or product for application to a plant or plant seed or on the surface of a plant or seed or in a plant sample or seed wash at a specific point in time, or over a period of time, that can be specific to a particular microbial species or strain, can be multiplexed for more than one strain, and/or can be performed more directly and without prior DNA extraction or significant purification steps.

### SUMMARY

This section provides a general summary of the disclosure and is not a comprehensive disclosure of its full scope or all of its features.

New and useful compositions for use in identifying and/or quantifying one or more bacterial and/or fungal and/or viral strain(s) are set forth herein. Methods for using the compositions to identify and/or quantify one or more bacterial and/or fungal and/or viral strain(s) are also set forth in the appended claims. Illustrative embodiments are further provided herein to enable a person skilled in the art to make and use the claimed subject matter.

In one aspect of the present disclosure, a composition for use in identifying and/or quantifying one or more bacterial and/or fungal and/or viral strain(s) is described. The composition generally includes one or more combination(s) of a forward primer sequence, a reverse primer sequence, and a probe sequence. Each of the one or more combination(s) can be specific for a single bacterial strain, a single viral strain, or a single fungal strain.

The forward and reverse primer sequences may be between about 15 and about 35 nucleotides in length or between about 20 and about 25 nucleotides in length. Additionally, or alternatively, the fragment between the forward and reverse primer sequences may be between about 50 and about 1000 nucleotides in length, between about 50 and about 500 nucleotides in length, between about 80 and about 400 nucleotides in length, between about 70 and about 180 nucleotides in length, or between about 120 and about 200 nucleotides in length, etc.

The probe sequence may be between about 15 and about 35 nucleotides in length, or between about 23 and about 30 nucleotides in length. The probe may include a fluorescent label, such as (without limitation) fluorescein amidite (FAM), VIC™, TAMRA™, HEX™, CY3™, CY5™, and JOE™.

The combination of a forward primer sequence, a reverse primer sequence, and a probe sequence may be selected from the following example combinations: (i) Combination 1 including SEQ ID NOs: 1-3, (ii) Combination 2 including SEQ ID NOs:4-6, (iii) Combination 3 including SEQ ID NOs:7-9, (iv) Combination 4 including SEQ ID NOs: 10-12, (v) Combination 5 including SEQ ID NOs:13-15, (vi) Combination 6 including SEQ ID NOs:16-18, or combinations thereof. Each of Combinations 1-5 may be used for identifying and/or quantifying bacterial strain NRRL B-67746. Combination 6 may be used for identifying and/or quantifying bacterial strain NRRL B-21661. In some aspects, the composition may include one, two, or more combinations independently selected from Combination 1, Combination 2, Combination 3, Combination 4, Combination 5, and Combination 6. Each of the one, two, or more combinations may be specific for identifying at least one bacterial strain and/or at least one fungal strain.

The composition may also include a PCR master mix.

The composition may further include a biosample. For example, the biosample may be a seed, a seed wash, or a liquid formulation (e.g., where the biosample may include, may be suspected of including, or may not include bacteria, spores of bacteria, and/or fungi; etc.) Where the biosample is a seed, the seed may be from an agricultural crop. The seed may be selected from a corn seed, a soybean seed, a cotton seed, a canola seed, a rice seed, a wheat seed, a sorghum seed, an alfalfa seed, a sugarcane seed, a millet seed, a tomato seed, a potato seed, a cucumber seed, cabbage seed, a broccoli seed, a cauliflower seed, a raspberry seed, a blackberry seed, a pumpkin seed, a squash seed, a strawberry seed, and a combination thereof. The seed wash may be generated from a seed selected from a corn seed, a soybean seed, a cotton seed, a canola seed, a rice seed, a wheat seed, a sorghum seed, an alfalfa seed, a sugarcane seed, a millet seed, a tomato seed, a potato seed, a cucumber seed, cabbage seed, a broccoli seed, a cauliflower seed, a raspberry seed, a blackberry seed, a pumpkin seed, a squash seed, a strawberry seed, and a combination thereof.

The biosample may comprise a plurality of bacterial strains and/or fungal strains (e.g., for use in promoting efficacy of the biosample, etc.) Alternatively, the biosample may not contain any bacteria or fungus (e.g., whereby the compositions and methods herein may be used to confirm the same, etc.)

The one or more bacterial strains that may be included in the biosample may include (without limitation) strains selected from a genus including Bacillus, Bradyrhizobium, Paenibacillus, Pseudoacidovorax, Phytobacter, Pseudomonas, and Xanthomonas. Further, the one or more bacterial strains may include one or more of NRRL B-67746 and NRRL B-21661.

The one or more fungal strains that may be included in the biosample may include (without limitation) strains selected from a genus including Penicillium, Thricoderma, Clonostachys, Phytophthora, and Fusarium.

In an embodiment in which the biosample is a seed that includes bacterial and/or fungal spores or cells (e.g., due to seed treatment with a microbe that provide a beneficial property, such as fungicidal, pesticidal or growth promotion activities) or a seed wash therefrom, the seed may have been coated with the following concentration of colony forming units (“CFU”) of bacterial and/or fungal spores or cells per seed: about 10 to about 1×10⁹ CFU/seed, about 1× 10² to about 1×10⁹ CFU/seed, about 1×10² to about 1×10⁹ CFU/seed, about 1×10³ to about 1×10⁹ CFU/seed, about 1×10⁴ to about 1×10⁹ CFU/seed, about 1×10⁵ to about 1×10⁹ CFU/seed, about 2.5×10⁵ to about 1×10⁹ CFU/seed, about 5×10⁵ to about 1×10⁹ CFU/seed, about 1×10⁵ to about 1×10⁸ CFU/seed, about 1×10⁵ to about 1×10⁷ CFU/seed, about 1×10⁶ CFU/seed, etc. In some examples, the biosample includes multiple such seeds, where each seed includes bacterial and/or fungal spores in the above concentration(s).

Further, the biosample may include one or more of a pesticide, an insecticide, a fungicide, a herbicide, or a combination thereof, for example, to promote efficacy of the biosample, etc. In addition, or alternatively, the biosample may include antibacterial compounds, polymers, colorants, dyes, etc.

In connection therewith, the biosample may not be subjected to DNA extraction, isolation and/or purification prior to or in connection with identifying and/or quantifying a bacterial strain or fungal strain or viral strain included in the biosample. Similarly, an assay sample created from the biosample may not be subjected to DNA extraction, isolation and/or purification prior to or in connection with identifying and/or quantifying a bacterial strain or fungal strain or viral strain included in the biosample prior to analysis. As such, the present disclosure provides unique assays that allow for quantification despite additional compounds, etc. included therein (e.g., compounds that may interfere with DNA amplification, etc.)

In another aspect of the present disclosure, a method for identifying and/or quantifying one or more bacterial and/or fungal strain(s) in a biosample is described herein. The method generally includes the steps of: (a) obtaining a biosample; (b) preparing an assay sample comprising all or part of the biosample; (c) performing an assay directly on the assay sample, or a portion thereof; (d) evaluating the results of the assay; (e) identifying one or more bacterial and/or fungal strain(s) as present in the biosample when the results are consistent with the presence of the one or more bacterial and/or fungal strain(s); and (f) quantifying an equivalent colony-forming unit (CFU) value for the one or more bacterial and/or fungal strain(s) present in the biosample, as identified in step (e), by comparing the results of step (d) with a standard curve for the one or more bacterial and/or fungal strain(s). The assay in the method may be quantitative PCR (qPCR), for example, digital qPCR. The qPCR may be performed directly on the assay sample without isolating and/or purifying and/or concentrating and/or enriching DNA from the biosample. The qPCR may utilize one or more combination(s) of a forward primer sequence, a reverse primer sequence, and a probe sequence specific for at least one known bacterial and/or fungal strain. The combination of a forward primer sequence, a reverse primer sequence, and a probe sequence may be selected from: (i) Combination 1 including SEQ ID NO:1, SEQ ID NO:2, and SEQ ID NO:3; (ii) Combination 2 including SEQ ID NO:4, SEQ ID NO:5, and SEQ ID NO:6; (iii) Combination 3 including SEQ ID NO:7, SEQ ID NO:8, and SEQ ID NO:9; (iv) Combination 4 including SEQ ID NO:10, SEQ ID NO:11, and SEQ ID NO:12; (v) Combination 5 including SEQ ID NO:13, SEQ ID NO: 14, and SEQ ID NO:15; (vi) Combination 6 including SEQ ID NO: 16, SEQ ID NO:17, and SEQ ID NO:18, and combinations thereof. In some aspects, the qPCR may utilize one, two, or more combinations of a forward primer sequence, a reverse primer sequence, and a probe sequence specific for at least one known bacterial and/or fungal strain. The one, two, or more combinations may be independently selected from Combination 1, Combination 2, Combination 3, Combination 4, Combination 5, and Combination 6.

The assay sample prepared in the method may further include the one or more combination(s) of a forward primer sequence, a reverse primer sequence, and a probe sequence specific for at least one known bacterial and/or fungal strain (e.g., where preparing the assay sample may further include combining the one or more combination(s) of the forward primer sequence, the reverse primer sequence, and the probe sequence specific for at least one known bacterial and/or fungal strain with the biosample or a portion thereof; etc.)

In the method of this aspect, step (d) may include calculating a cycle threshold (Ct) value for the assay sample. As used herein, the Ct value represents the number of qPCR cycles it takes for a signal (e.g., fluorescent signal, etc.) to cross a base/threshold line during DNA amplification.

In the method of this aspect, step (e) may include identifying a first bacterial and/or fungal strain as present in the biosample based on a first fluorescent signal detected for the assay sample satisfying a first background value, and identifying a second different bacterial and/or fungal strain as present in the biosample based on a second fluorescent signal detected for the assay sample satisfying a second background value. The first bacterial and/or fungal strain may be specific to a first one of the two or more combinations, and the second bacterial and/or fungal strain may be specific to a second one of the two or more combinations. The first background value may be the same as the second background value. Additionally, step (e) may include identifying the one or more bacterial and/or fungal strain(s) as present in the biosample based on a fluorescent signal detected for the assay sample satisfying at least one background value.

In the method of this aspect, step (f) may include quantifying an equivalent CFU value for each of the one or more bacterial and/or fungal strain(s) present in the biosample as identified in step (e) of the method, by comparing the Ct value of step (d) with the standard curve for each of the one or more bacterial and/or fungal strain(s). The standard curve for each of the one or more bacterial and/or fungal strain(s) may describe a correlation between the Ct value and the equivalent CFU value for each of the one or more bacterial and/or fungal strain(s).

In connection with the above, quantifying and/or identifying the presence and/or amount of a certain microbe or the relative presence and/or amounts of a combination of microbes in a formulation or product may be determined via CFU counting, qPCR, etc., as described above. Alternatively, for example, a standard/reference curve may be generated via CFU counting for a single microbial species to be compared to the results of a qPCR assay.

Additionally, or alternatively, the method may include distinguishing microbial load amounts on seed samples. For example, the Ct value of step (d) may be compared to a CFU standard curve for each of the one or more bacterial and/or fungal strain(s) to quantify the CFU load amount. The method may indicate the CFU load applied to a seed sample. The method may be used for CFU load quality control.

The biosample assayed by the method of this aspect may be a seed, a seed wash, or a liquid formulation (e.g., where the biosample may include, may be suspected of including, or may not include bacteria, spores of bacteria, and/or fungi; etc.) The seed may be selected from (without limitation) a corn seed, a soybean seed, a cotton seed, a canola seed, a rice seed, a wheat seed, a sorghum seed, an alfalfa seed, a sugarcane seed, a millet seed, a tomato seed, a potato seed, a cucumber seed, cabbage seed, a broccoli seed, a cauliflower seed, a raspberry seed, a blackberry seed, a pumpkin seed, a squash seed, a strawberry seed, and a combination thereof. The seed wash may be generated from a seed selected from a corn seed, a soybean seed, a cotton seed, a canola seed, a rice seed, a wheat seed, a sorghum seed, an alfalfa seed, a sugarcane seed, a millet seed, a tomato seed, a potato seed, a cucumber seed, cabbage seed, a broccoli seed, a cauliflower seed, a raspberry seed, a blackberry seed, a pumpkin seed, a squash seed, a strawberry seed, and a combination thereof.

Where the biosample is a seed, the method may include combining the seed with a solvent and agitating the seed and solvent together to prepare the assay sample. The solvent may include PBS, a surfactant, or a combination thereof. Alternatively, preparing the assay sample may coincide with obtaining the biosample, whereby the assay sample is the biosample (or is a portion of the biosample), for example, without further processing of the biosample.

The method may further include serially diluting the assay sample, or a portion thereof. The assay may then be performed directly on the serially diluted assay sample. Additionally, or alternatively, the method may include serially diluting the biosample, or a portion thereof. And, the assay may then be performed directly on the serially diluted biosample, or portion thereof, as the assay sample.

The assay may be performed on the assay sample in triplicate. Multiple analyses may be carried out from one biosample. For example, the assay may include multiple qPCR reactions to quantify/identify different microbes.

In the method of this aspect, step (e) may include identifying one or more bacterial strain(s) as present in the biosample, wherein the identified one or more bacterial strain(s) includes bacterial spores present in the biosample. The bacterial spores may be gram positive or gram negative. A genus of the one more bacterial strain(s) may include (without limitation) Bacillus, Bradyrhizobium, Paenibacillus, Pseudoacidovorax, Phytobacter, Pseudomonas, and Xanthomonas. Further, the one or more bacterial strain(s) may include one or more of NRRL B-67746 and NRRL B-21661.

The assay may be performed on the assay sample in triplicate.

In the method of this aspect, step (e) may include identifying one or more fungal strain(s) as present in the biosample. A genus of the one or more fungal strain(s) may include (without limitation) Penicillium, Thricoderma, Clonostachys, Phytophthora, and Fusarium.

Further, the biosample may include one or more of a pesticide, an insecticide, a fungicide, or a combinations thereof, for example, to promote efficacy of the biosample, etc.

The method of this aspect may further include determining that the biosample is viable based on the equivalent CFU value of step (f) satisfying a viability threshold value.

In some embodiments herein, the assay performed on the assay sample as part of the method of this aspect may utilize at least two combinations of primer and probe sequences, including a first combination and a second combination. The first combination may include a first forward primer sequence, a first reverse primer sequence, and a first probe sequence, and the second combination may include a second forward primer sequence, a second reverse primer sequence, and a second probe sequence. The first combination and second combination may each be specific to different bacterial and/or fungal strains. Identifying one or more bacterial and/or fungal strain(s) as present in the biosample, for example, at step (e), may then include differentially identifying at least two bacterial and/or fungal strains as present in the biosample.

In such aspects, quantifying an equivalent CFU value for the at least two bacterial and/or fungal strains present in the biosample may include quantifying an equivalent CFU value for each of the at least two bacterial and/or fungal strains present in the biosample. The method may further include, then, determining if the at least two bacterial and/or fungal strains present in the biosample are viable based on the equivalent CFU value, for example, of step (f) for each of the at least two bacterial and/or fungal strains present in the biosample satisfying one or more viability threshold value(s).

The biosample may be stored for a period of time prior to obtaining the biosample, for example, at least about six months or at least about 1 year. In addition, the method of this aspect, steps (b)-(f) may be performed within a specified time period of obtaining the biosample. For example, the specified time period may be about twelve hours or less, about eight hours or less, about six hours or less, about four hours or less, about two hours or less, etc.

The method of this aspect may further include incubating the biosample or assay sample with a dye that inhibits nucleotide amplification. The dye may intercalate a nucleotide sequence. The dye may be selected from (without limitation) Propidium Monoazide, Ethidium Monoazide or modified forms of these dyes. Alternatively, the method may not include a probe sequence.

Further, the biosample may be a first biosample obtained from a bulk supply and the method may include obtaining the first biosample from a bulk supply. The method may further include obtaining a second biosample from the same or different bulk supply and repeating steps (b)-(f) for the second biosample. Obtaining the first biosample from the bulk supply may include obtaining the first biosample from the bulk supply at a first time. The second biosample may be obtained from the bulk supply at a second time at least one or more day(s) after the first time. The method may further include repeating steps (b)-(f) for the second biosample, which may include performing steps (c)-(f) for the second biosample at least one or more day(s) after performing steps (c)-(f) for the first biosample.

Still further in the method of this aspect, the biosample may include a seed (or multiple seeds) that has been treated with one or more bacteria or fungi, such that the multiple bacteria and/or fungi are disposed as a coating on the seed. In connection therewith, then, identifying one or more bacterial and/or fungal strain(s) as present in the biosample includes identifying the bacteria and/or fungi as present in the coating on the seed(s). In some examples, the multiple bacteria and/or fungi applied to the seed(s) as the treatment may include between about 1×10² CFU of each bacteria or fungi per seed to about 1×10⁷ CFU of each bacteria or fungi per seed.

In another aspect of the present disclosure, a method for identifying and/or quantifying one or more bacteria and/or fungus of a biosample is provided. The biosample may include a seed or a liquid formulation configured as a treatment for a seed. The method then generally includes obtaining the biosample from a bulk supply of the seed or from a bulk supply of the liquid formulation, contacting (e.g., rinsing, soaking, mixing, etc.) the biosample with a quantity of a solvent to prepare an assay sample, performing qPCR directly on the assay sample, or a portion thereof, without isolating and/or purifying and/or concentrating and/or enriching DNA from the biosample, and calculating a cycle threshold (Ct) value for the assay sample. The biosample may further include multiple bacterial and/or fungal spores and one or more of a pesticide, an insecticide, and/or a fungicide. The method may further include identifying the bacterial and/or fungal spores as present in the biosample, and quantifying the bacterial and/or fungal spores present in the biosample as an equivalent colony-forming unit (CFU) value by comparing the Ct value with a standard curve for the bacterial and/or fungal spores.

Further areas of applicability will become apparent from the description provided herein. The description and specific examples in this summary are intended for purposes of illustration only and are not intended to limit the scope of the present disclosure.

### BRIEF DESCRIPTION OF THE DRAWINGS

The drawings described herein are for illustrative purposes only of selected embodiments and not all possible implementations, and are not intended to limit the scope of the present disclosure.

FIG. 1 illustrates an example method of the present disclosure for identifying and quantifying one or more bacterial and/or fungal strain(s) in a biosample;

FIG. 2 illustrates qPCR reference curves for Bacillus velezensis (NRRL B-67746);

FIG. 3 illustrates qPCR reference curves for liquid samples containing Bacillus amyloliquefaciens (NRRL B-21661) spores;

FIGS. 4A-4B illustrate single species samples tested against either their specific primers and probe or against the primers and probes for the other species, where FIG. 4A illustrates fluorescence signals with a Ser (Combination 6) primer and probe set detecting only B. amyloliquefaciens and FIG. 4B illustrates fluorescence signals with a 32-5 (Combination 1) primer and probe set detecting only B. velezensis;

FIGS. 5A-5B illustrate mixed spore samples tested against primers and probe sets 32-5 or Ser, where FIG. 5A illustrates reactions against 32-5 set (Combination 1) and FIG. 5B illustrates reactions against Ser set (Combination 6);

FIG. 6 illustrates qPCR curves for different PCR mixes, as evaluated for both on-seed samples and colony plate samples of B. velezensis;

FIG. 7 illustrates qPCR curves for different CFU loads of B. velezensis; and

FIG. 8 illustrates qPCR reference curves for on seed samples where the reactions were set up in a 384-well plate format.

Corresponding reference numerals indicate corresponding parts throughout the drawings and description herein.

### SEQUENCE LISTING

The nucleotide sequences listed in the accompanying sequence listing are shown using standard letter abbreviations for nucleotide bases. The nucleotide sequences follow the standard convention of beginning at the 5′ end of the sequence and proceeding forward (i.e., from left to right in each line) to the 3′ end. Only one strand of each nucleic acid sequence is shown, but (unless otherwise stated) the complementary strand is understood to be included by reference to the displayed strand.

SEQ ID NO:1 sets forth the nucleotide sequence of the forward primer sequence of Combination 1: CAATTACCTT TACCTGTCAC AAGC.

SEQ ID NO:2 sets forth the nucleotide sequence of the reverse primer sequence of Combination 1: AAGCCATTTC TTGCATTCTT CC.

SEQ ID NO:3 sets forth the nucleotide sequence with fluorescent label of the probe sequence of Combination 1: 6-FAM/AGAGGATAT/ZEN/G ACACATGAAG AACTGCCA.

SEQ ID NO:4 sets forth the nucleotide sequence of the forward primer sequence of Combination 2: GCAACAAGTC TTTCTCGTTC TG.

SEQ ID NO:5 sets forth the nucleotide sequence of the reverse primer sequence of Combination 2: GACTGGGTTT CTAGCAGAGA AG.

SEQ ID NO:6 sets forth the nucleotide sequence with fluorescent label of the probe sequence of Combination 2: 6-FAM/CCTCGATAA/ZEN/A CTTCTCTGTG CCATCAGG.

SEQ ID NO:7 sets forth the nucleotide sequence of the forward primer sequence of Combination 3: ACCTCGATAA ACTTCTCTGT GC.

SEQ ID NO:8 sets forth the nucleotide sequence of the reverse primer sequence of Combination 3: CTAAGATGGT TGACTGGGTT TCT.

SEQ ID NO:9 sets forth the nucleotide sequence with fluorescent label of the probe sequence of Combination 3: 6-FAM/TCATAGCCA/ZEN/T GTCCATCTTC TCTGCT.

SEQ ID NO:10 sets forth the nucleotide sequence of the forward primer sequence of Combination 4: TGGTAAGCTT CGATCAGAA.

SEQ ID NO:11 sets forth the nucleotide sequence of the reverse primer sequence of Combination 4: CACTGGTAAG TGATGCAGTG AAA.

SEQ ID NO: 12 sets forth the nucleotide sequence with fluorescent label of the probe sequence of Combination 4: 6-FAM/ACATCCTCC/ZEN/G GTTCTCCTTC AGT.

SEQ ID NO:13 sets forth the nucleotide sequence of the forward primer sequence of Combination 5: CTGTATATCT AAACCCTGGA ATCTCTT.

SEQ ID NO:14 sets forth the nucleotide sequence of the reverse primer sequence of Combination 5: GTTAGAAACA TGCAAGATGG TCAG.

SEQ ID NO: 15 sets forth the nucleotide sequence with fluorescent label of the probe sequence of Combination 5: 6-FAM/TGGCTGAAG/ZEN/T CAAAGGGCCA ATTG.

SEQ ID NO:16 sets forth the nucleotide sequence of the forward primer sequence of Combination 6: GACGTATGGA TACACCTCTT TAAT.

SEQ ID NO:17 sets forth the nucleotide sequence of the reverse primer sequence of Combination 6: CCAAATTCCT CAGAAGAGAG AG.

SEQ ID NO: 18 sets forth the nucleotide sequence with fluorescent label of the probe sequence of Combination 6: 6-FAM/TTCCCATTA/ZEN/A TATACTCAAT TAGAGAACCT.

### DETAILED DESCRIPTION

Example embodiments will now be described more fully with reference to the accompanying drawings. The description and specific examples included herein are intended for purposes of illustration only and are not intended to limit the scope of the present disclosure.

Determining whether a seed is coated with a microbe or whether a liquid fertilizer composition contains microbes has traditionally been a time-consuming process. For instance, samples derived from a seed or liquid fertilizer composition can be cultured, e.g., on agar plates, and the microbes can grow on or in said culture. DNA from the cultured microbes can then be extracted, cleaned, and analyzed by quantitative PCR (qPCR).

The compositions and methods of the present disclosure provide, for example, manners for identifying and quantifying (and/or quantitating) an amount of one or more bacterial and/or fungal and/or viral strains on a plant or seed (e.g., an agricultural plant or seed, etc.) or within a seed wash or in a liquid formulation. In doing so, such identification and quantification of the bacterial and/or fungal strains can allow for the sowing of seeds that are properly coated with the appropriate type and amount of microbes to promote plant growth and yield. Such identification and quantification may also allow for making confirmations that plants, seeds, and/or liquid formulations do or don't include certain bacterial and/or fungal strains, for example, as part of quality control operations, etc. Further, such identification and quantification (and/or quantitation) may allow for making viability determinations for products (e.g., plants, seeds, liquid formulations, etc.) including the particular identified bacterial and/or fungal strains.

### I. Definitions

In the context of this disclosure, a number of terms and abbreviations are used. The following definitions are provided.

As used herein, the term “plant” may refer to any living organism belonging to the kingdom Plantae (i.e., any genus/species in the Plant Kingdom). For example, the term “plant” can encompass a tree, herb, bush, grass, vine, fern, moss, or green algae. The plant may be monocotyledonous (monocot), or dicotyledonous (dicot).

Examples of plants may include, but are not limited to, Arabidopsis, Brachypodium, switchgrass, rose, sunflower, bananas, opo, pumpkins, squash, lettuce, cabbage, oak trees, guzmania, geraniums, hibiscus, clematis, poinsettias, sugarcane, taro, duck weed, pine trees, Kentucky blue grass, zoysia, coconut trees, cauliflower, cavalo, collards, kale, kohlrabi, mustard greens, rape greens, and other brassica leafy vegetable crops, bulb vegetables (e.g., garlic, leek, onion (dry bulb, green, and Welch), shallot, etc.), citrus fruits (e.g., grapefruit, lemon, lime, orange, tangerine, citrus hybrids, pummelo, etc.), cucurbit vegetables (e.g., cucumber, citron melon, edible gourds, gherkin, muskmelons (including hybrids and/or cultivars of Cucumis melons), watermelon, cantaloupe, and other cucurbit vegetable crops), fruiting vegetables (including eggplant, ground cherry, pepino, pepper, tomato, tomatillo), grape, leafy vegetables (e.g., romaine, etc.), root/tuber vegetables (e.g., potato, etc.), and tree nuts (almond, pecan, pistachio, and walnut), berries (e.g., tomatoes, barberries, currants, elderberries, gooseberries, honeysuckles, mayapples, nannyberries, Oregon-grapes, see-buckthoms, hackberries, bearberries, lingonberries, strawberries, sea grapes, blackberries, cloudberries, loganberries, raspberries, salmonberries, thimbleberries, and wineberries, etc.), cereal crops (e.g., corn (maize), rice, wheat, barley, sorghum, millets, oats, ryes, triticales, buckwheats, fonio, quinoa, oil palm, etc.), Brassicaceae family plants, Fabaceae family plants, pome fruit (e.g., apples, pears), stone fruits (e.g., coffees, jujubes, mangos, olives, coconuts, oil palms, pistachios, almonds, apricots, cherries, damsons, nectarines, peaches and plums, etc.), vine (e.g., table grapes, wine grapes, etc.), fiber crops (e.g., hemp, cotton, etc.), ornamentals, and the like.

As used herein the term “biosample” may encompass or may be applied to or may be present on a plant, multiple plants, a seed, multiple seeds, or a portion of the plant(s) or seed(s). The biosample may include a plant wash or a seed wash, wherein the plant or seed, or a portion thereof, is placed in a solvent and/or rinsed with a solvent. The solvent recovered following incubation or rinsing represents a plant wash or seed wash, as appropriate. For purposes of this disclosure, the biosample is not cultured or otherwise grown nor are the cells within the biosample lysed or otherwise manipulated to isolate, extract, purify, and/or concentrate nucleotides (e.g., DNA or RNA, etc.). Instead, the biosample, or a portion or aliquot thereof, is placed directly in an assay medium, or an assay medium is added directly to the biosample, to perform an assay directly on the biosample or assay sample, or a portion thereof. A biosample may comprise other components that may be part of a formulation applied to a plant or seed or may be used to obtain plant or seed wash from a plant or plant seed, such as, for example, one or more salts, buffers, surfactants, excipients, nutrients, pesticides, insecticides, fungicides, fertilizers, plant growth regulators, other biological agents or additives, etc., or combinations thereof.

Additionally, the term “biosample” may encompass a liquid formulation that may be sprayed on or otherwise provided to plants and/or seeds, or added to soil that contains or will contain the plants or seeds. The liquid formulation may comprise, or is suspected of comprising bacteria, spores of bacteria, or fungi. Alternatively, the liquid formulation may be sterile (e.g., it comprises none of bacteria, spores of bacteria, or fungi, etc.) or be suspected of being sterile.

For the compositions and methods described herein, the biosample can be directly assayed or an assay sample can be prepared from the biosample. As used herein, the term “assay sample” may refer to a composition comprising the biosample that is not manipulated or minimally manipulated prior to an assay and is ready for use in said assay. The assay sample may represent a composition comprising a biosample in an assay medium (e.g., a buffer and possibly other components, such as a surfactant, salts, nutrients, etc.) Alternatively or additionally, the assay sample may represent a serial dilution of the biosample into increasingly dilute samples. The assay sample is not further manipulated to lyse cells or isolate, extract, purify, enrich, and/or concentrate nucleotides (e.g., DNA or RNA, etc.) contained within the biosample prior to amplification.

As used herein, the term “bacteria” may refer to unicellular prokaryote microorganisms and spores thereof. The term is intended to encompass all microorganisms that are considered bacteria, including mycoplasma. All forms of bacteria are encompassed herein, including cocci, bacilli, spirochetes, spheroplasts, protoplasts, and the like. The term also includes gram-negative and gram-positive bacteria as well as spore-forming bacteria and non-spore-forming bacteria. A “bacterial strain” refers to a genetic variant, isoform, and/or subtype of a bacterial species.

As used herein, the term “fungus” may refer to eukaryotic microorganisms from the kingdom Fungi, such as molds, yeasts, and mushrooms, and includes spores thereof. A “fungal strain” refers to a genetic variant, isoform, and/or subtype of a fungal species.

As used herein, the term “plant wash” may refer to a composition, e.g., a solvent, that may be used to rinse and/or wash one or more plants or one or more parts of one or more plants. The plant wash is configured to collect, suspend, or otherwise remove bacteria and/or fungi, or spores of the bacteria and/or fungi, from the plant. The plant wash can be directly assayed or further manipulated to be in an assay sample.

As used herein, the term “seed wash” may refer to a composition, e.g., a solvent, that may be used to rinse and/or wash seeds. The seed wash is configured to collect, suspend, or otherwise remove bacteria and/or fungi, or spores of the bacteria and/or fungi, from the seed. The seed wash can be directly assayed or further manipulated to be in an assay sample.

As used herein, the terms “pesticide”, “insecticide”, “herbicide” and “fungicide” may refer to compounds, biomolecules, or agents that kill or inhibit the growth of pests, insects, plants and fungi, respectively. The pesticide, insecticide, herbicide and fungicide may be specific for one or a few pests, insects, plants and fungi, or may be general (e.g., wide spectrum, etc.) and kill or inhibit the growth of many pests, insects, plants or fungi. In some embodiments, the same composition may exhibit any combination of pesticidal, insecticidal, herbicidal and fungicidal properties.

As used herein, the term “primer” may refer to a nucleotide sequence, comprised of either DNA or RNA, or a mixture of the two, that recognizes a DNA sequence, such as bacterial or fungal DNA. The DNA sequence recognized by the primers described herein may be specific to one microbial species. The bacterial or fungal DNA may be genomic or non-genomic DNA. The primer sequences may be between about 15 and about 35 nucleotides in length. In an embodiment, the primer may be designated as a “forward primer” or “forward primer sequence.” Alternatively, the primer may be designated as a “reverse primer” or “reverse primer sequence.” The terms “forward” and “reverse” refer to the relative orientation of the primer sequences to a bacterial or fungal DNA template, are generally separated by between about 50 and about 1000 nucleotides and are generally used in combination. As disclosed below, the forward and reverse primer sequences are included in the same composition, typically along with a probe sequence.

As used herein, the term “probe” or “probe sequence” may refer to a nucleotide sequence comprised of either DNA or RNA, or a mixture of the two that recognizes a DNA sequence, such as bacterial or fungal DNA sequence.

### II. Compositions

Compositions are provided herein that include one or more combination(s) of a forward primer sequence, a reverse primer sequence, and a probe sequence (e.g., one or more of the Combinations 1-6 described herein, etc.) The compositions may be used for identifying and/or quantifying one or more bacterial and/or fungal strain(s), for example, in a biosample, etc. In connection therewith, the one or more combination(s) included in the compositions may be specific for a single bacterial strain or a single fungal strain (or not).

In any embodiment, the compositions provided herein may also include a biosample. For example, the biosample may be a seed or may be a seed wash. The seed or seed wash generated therefrom, may be from an agricultural crop. Examples of a seed include, but are not limited to, a corn seed, a soybean seed, a cotton seed, a canola seed, a rice seed, a wheat seed, a sorghum seed, an alfalfa seed, a sugarcane seed, a millet seed, a tomato seed, a potato seed, a cucumber seed, a cabbage seed, a broccoli seed, a cauliflower seed, a raspberry seed, a blackberry seed, a pumpkin seed, a squash seed, a strawberry seed, and combinations thereof.

The seed wash may be generated from but not limited to any of the above described seeds. The seed wash may include a solvent, for example, an aqueous composition comprising water and, optionally, additional components. The additional components include, but are not limited to, salts (including phosphate, chloride, bromide, iodide, amino, sodium, potassium, magnesium, and calcium salts), surfactants (e.g., nonionic surfactants), detergents, acidic agents, and alkaline agents. In any embodiment, the solvent may include a buffer, such as phosphate-buffered saline (PBS). For example, the solvent may include PBS and polyoxyethylene (20) sorbitan monooleate (commercially available as TWEEN® 80). Without limitation, in example embodiments the seed wash may have a pH ranging from about 6.5 to about 8.0. For example, the pH of the resulting seed wash may be about 7.4.

Additionally or alternatively, a biosample may comprise a plant or plant part. The plant or part thereof, again, may be selected from corn, soybean, cotton, canola, rice, wheat, sorghum, alfalfa, sugarcane, millet, tomato, potato, cucumber, cabbage, broccoli, cauliflower, raspberry, blackberry, pumpkin, squash, strawberry, and combinations thereof (and any other plant or part thereof identified herein).

Additionally or alternatively, a biosample may be or may comprise a liquid formulation or solution, for example, that may be sprayed on or otherwise provided to a plant and/or seed (e.g., without limitation, formulations of Bacillus spp., formulations for P. bilaiae, etc.)

In any embodiment, the biosample may include one or more bacterial strains, fungal strains (e.g., a plurality of bacterial strains and/or fungal strains), or a combination thereof. For example, a genus of the one or more bacterial strains may include (without limitation) Bacillus, Bradyrhizobium, Paenibacillus, Pseudoacidovorax, Phytobacter, Pseudomonas, Xanthomonas, and combinations thereof. In another embodiment, the bacteria may include (without limitation) B. velezensis, B. amyloliquefaciens, B. subtilis, and combinations thereof. Further, the bacterial strain may include NRRL B-67746, NRRL B-21661, and combinations thereof.

A genus of the one or more fungal strains may include (without limitation) Penicillium, Trichoderma, Clonostachys, Phytophthora, Fusarium, and combinations thereof.

In alternative embodiments, the biosample may not contain specific bacterial strains, fungal strains, or a combination thereof. In such embodiments, the methods herein may be employed to confirm the same, i.e., that the biosample does not contain specific bacterial strains, fungal strains, or combinations thereof (e.g., as part of quality control operations, etc.)

Additionally or alternatively, in some embodiments the biosample may further include a pesticide, an insecticide, a fungicide, or combinations thereof.

As described above, bacterial or fungal spores or cells may be added to one or more seeds, for example, as a seed treatment, etc. In some examples, the CFU of microbes per seed may be greater than or equal to about 10 CFU/seed, greater than or equal to about 1× 10² CFU/seed, greater than or equal to about 1×10³ CFU/seed, greater than or equal to about 1×10⁴ CFU/seed, greater than or equal to about 1×10⁵ CFU/seed, greater than or equal to about 1×10⁶ CFU/seed, greater than or equal to about 1×10⁷ CFU/seed, greater than or equal to about 1×10⁸ CFU/seed, about 10 to about 1×10⁸ CFU/seed, about 1×10² to about 1×10⁸ CFU/seed, about 1×10³ to about 1×10⁸ CFU/seed, about 1×10⁴ to about 1×10⁷ CFU/seed, about 1×10⁵ to about 1×10⁷ CFU/seed, about 1×10⁵ to about 1×10⁸ CFU/seed, about 1×10⁶ to about 1×10⁸ CFU/seed, about 1×10⁶ to about 1×10⁷ CFU/seed, about 10 to about 3×10⁶ CFU/spores, about 1×10⁶ CFU/seed, etc.

Additionally, the bacterial and/or fungal microbes may be included in a liquid formulation. In some example embodiments, the concentration of microbes in the liquid formulation may be greater than or equal to about 1×10³ CFU/mL, greater than or equal to about 1×10⁴ CFU/mL, greater than or equal to about 1×10⁵ CFU/mL, greater than or equal to about 1×10⁶ CFU/mL, greater than or equal to about 1×10⁷ CFU/mL, greater than or equal to about 1×10⁸ CFU/mL, greater than or equal to about 1×10⁹ CFU/mL, greater than or equal to about 1×10¹⁰ CFU/mL, greater than or equal to about 1×10¹¹ CFU/mL, greater than or equal to about 1×10¹² CFU/mL, about 1×10⁵ to about 1×10¹² CFU/mL, about 1×10⁶ to about 1×10¹² CFU/mL, about 1×10⁷ to about 1×10¹² CFU/mL about 1×10⁸ to about 1×10¹² CFU/mL, or about 1×10¹⁰ CFU/mL. A liquid formulation of microbes may be tested upon production and then at a later timepoint(s) to test for microbial stability in the formulation.

In some example embodiments, the spore concentration per seed (as generally described above) may be independent of the seed's shape, type (e.g., soy, maize, etc.), volume, surface area, size, etc.

In some embodiments, the biosample comprises DNA that is not isolated and/or purified prior to or in connection with identifying and/or quantifying a bacterial strain and/or fungal strain that is present in the biosample. In connection therewith, in some embodiments, an assay as described herein may be performed directly on the biosample or a portion thereof (e.g., without additional processing, or with minimal processing, of the biosample in preparation for performing the assay (whereby the biosample may represent a dirty assay sample, etc.), etc.)

In any embodiment, the forward and reverse primer sequences independently may be between about 15 and about 35 nucleotides in length or between about 20 and about 25 nucleotides in length. For example, the forward and reverse primer sequences independently may be about 15, about 16, about 17, about 18, about 19, about 20, about 21, about 22, about 23, about 24, about 25, about 26, about 27, about 28, about 29, about 30, about 31, about 32, about 33, about 34, or about 35 nucleotides in length. The forward primer sequence may include SEQ ID NO:1, SEQ ID NO:4, SEQ ID NO:7, SEQ ID NO:10, SEQ ID NO:13, and SEQ ID NO: 16. The reverse primer sequence may include SEQ ID NO:2, SEQ ID NO:5, SEQ ID NO:8, SEQ ID NO: 11, SEQ ID NO: 14, and SEQ ID NO: 17.

Additionally or alternatively, the fragment between the forward and reverse primer sequences may be between about 50 and about 1000 nucleotides, between about 50 and about 500 nucleotides, between about 120 and about 200 nucleotides, between about 70 and about 180 nucleotides, or between about 80 and about 400 nucleotides. For example, the fragment between the forward and reverse primer sequences may be about 50, about 100, about 150, about 200, about 250, about 300, about 350, about 400, about 450, about 500, about 550, about 600, about 650, about 700, about 750, about 800, about 850, about 900, about 950, or about 1000 nucleotides.

In any embodiment, the probe sequence may be between about 15 and about 35 nucleotides in length, or between about 23 and about 30 nucleotides in length. For example, the probe sequences may be about 15, about 16, about 17, about 18, about 19, about 20, about 21, about 22, about 23, about 24, about 25, about 26, about 27, about 28, about 29, about 30, about 31, about 32, about 33, about 34, or about 35 nucleotides in length. The probe sequence may include SEQ ID NO:3, SEQ ID NO:6, SEQ ID NO:9, SEQ ID NO: 12, SEQ ID NO:15, and SEQ ID NO:18.

In some embodiments, the probe sequence may include a fluorescent label. Any suitable fluorescent label may be used. For example, the fluorescent label may include (without limitation) a fluorescein amidite (FAM), Aequorea victoria green fluorescent protein (VIC™), 5-carboxytetramethylrhodamine (TAMRA™), hexachloro-fluorescein (HEX™), 2-[3-[1-[6-[(2,5-dioxo-1-pyrrolidinyl)oxy]-6-oxohexyl]-1,3-dihydro-3,3-dimethyl-5-sulfo-2H-indol-2-ylidene]-1-propen-1-yl]-1-ethyl-3,3-dimethyl-5-sulfo-3H-indolium (CY3™), 2-[5-[1-[6-[(2,5-dioxo-1-pyrrolidinyl)oxy]-6-oxohexyl]-1,3-dihydro-3,3-dimethyl-5-sulfo-2H-indol-2-ylidene]-1,3-pentadien-1-yl]-1-ethyl-3,3-dimethyl-5-sulfo-3H-indolium (CY5™), and 6-carboxy-4′,5′-dichloro-2′,7′-dimethoxyfluorescein succinimidyl ester (JOE™) and combinations thereof. Additionally or alternatively, the probe sequence may comprise a quencher, for example, to inhibit fluorescence signals, etc. Any suitable quencher may be used. For example, the quencher may include (without limitation) ZEN, Iowa Black, and TAO/lowa Black (all available from Integrated DNA Technologies). In an alternative embodiment, the probe sequence does not contain a fluorescent label.

In any embodiment, a forward primer sequence, a reverse primer sequence, and a probe sequence may be combined in a composition. Non-limiting combinations of a forward primer sequence, a reverse primer sequence, and a probe sequence are listed below in Table 1. The combination may be used for identifying and/or quantifying a bacterial strain, a fungal strain, or a combination thereof. For example, the bacterial strain identified by any of the combinations may be NRRL B-67746, NRRL B-21661, or a combination thereof. Additionally or alternatively, the fungal strain identified by any of the combinations may be Trichoderma, Clonostachys, Phytophthora, and Fusarium.

TABLE 1
Combinations

| Combination | Forward | Reverse | Probe |
|---|---|---|---|
| No. (Name) | Primer Seq. | Primer Seq. | Seq. |
| 1 (32-5) | SEQ ID NO: 1 | SEQ ID NO: 2 | SEQ ID NO: 3 |
| 2 (30-2) | SEQ ID NO: 4 | SEQ ID NO: 5 | SEQ ID NO: 6 |
| 3 (30-3) | SEQ ID NO: 7 | SEQ ID NO: 8 | SEQ ID NO: 9 |
| 4 (85-2) | SEQ ID NO: 10 | SEQ ID NO: 11 | SEQ ID NO: 12 |
| 5 (86L-2) | SEQ ID NO: 13 | SEQ ID NO: 14 | SEQ ID NO: 15 |
| 6 (Ser) | SEQ ID NO: 16 | SEQ ID NO: 17 | SEQ ID NO: 18 |

In any embodiment, the compositions herein may include only one of Combination 1, Combination 2, Combination 3, Combination 4, Combination 5, and Combination 6. In an alternative embodiment, the compositions herein may include two or more of Combination 1, Combination 2, Combination 3, Combination 4, Combination 5, and Combination 6. Additionally or alternatively, the compositions may include two or more combinations as described herein, wherein each of the two or more combinations may be specific for identifying at least one bacterial strain or at least one fungal strain.

The compositions herein may optionally comprise additional components. For example, the additional component may include a PCR master mix. As used herein, the PCR master mix generally comprises a DNA polymerase, deoxynucleotide triphosphates (dNTPs), MgCl2, and appropriate reaction buffers. The DNA polymerase and reaction buffers are not particularly limited. Any conventional or known thermo-resistant DNA polymerase and/or reaction buffer may be utilized. Additionally or alternatively, any of the PCR master mix components can be removed and/or replaced with other components known in the art to facilitate PCR (e.g., qPCR, etc.) reactions.

### III. Methods

Methods are provided herein for identifying and/or quantifying one or more bacterial and/or fungal strain(s) in a biosample. The methods may be implemented on any desired biosample, for example, as described herein, through use of the combinations described herein (e.g., through use of one or more of Combinations 1-6, etc.).

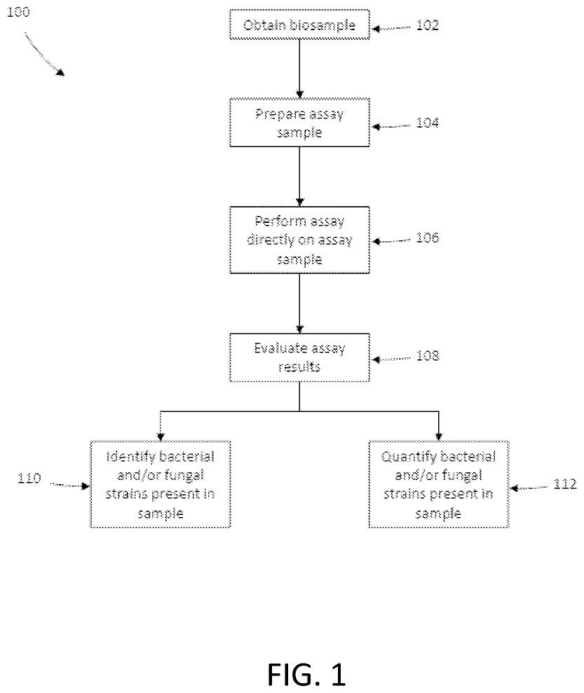

That said, FIG. 1 illustrates an example embodiment of a method 100 for identifying and/or quantifying one or more bacterial and/or fungal strain(s) in a biosample. The illustrated method 100 generally includes the steps of: (a) obtaining a biosample (102); (b) preparing an assay sample from all or part of the biosample (104); (c) performing an assay directly on the assay sample, or a portion of the assay sample (106); (d) evaluating the results of the assay (108); (e) identifying one or more bacterial and/or fungal strains as present in the biosample when the results are consistent with the presence of one or more bacterial and/or fungal strains (110); and (f) quantifying an equivalent colony-forming unit (CFU) value for the one or more bacterial and/or fungal strains present in the biosample (112).

As described above, the biosample may include a plant, a seed, or a portion of the plant or seed. Or, it may include a liquid formulation that may be applied, for example, on or to plants and/or seeds, or added to soil that contains the plants or seeds. In either case, obtaining the biosample (e.g., at 102 in the method 100, etc.) may include obtaining the plant, seed, liquid formulation, etc. immediately from a breeding program or otherwise, or immediately following preparation thereof (e.g., a liquid formulation, etc.), or from a bulk supply of the same. For instance, and as discussed above, the biosample may be stored for a period of time prior to obtaining the biosample (and/or prior to preparing the assay sample therefrom). For example, the biosample may be stored (after initial preparation thereof) for a period of time of at least 1 week, at least 2 weeks, at least 1 month, at least 3 months, at least six months, or at least one year or more.

In connection with the above, the biosample may include, or may be suspected of including, a genus of bacteria and/or a genus of fungi and/or a genus of virus (as generally described herein). As such, the biosample may be selected in order to confirm that the genus of bacteria and/or a genus of fungi and/or genus of virus is actually present and/or to confirm that the genus of bacteria and/or a genus of fungi and/or a genus of virus (if present) is viable. Additionally, in some example embodiments, the biosample may also include one or more of a pesticide, an insecticide, and/or a fungicide included therein (e.g., as part of a commercial product or commercial offering for seeds, liquid formulations, etc.)

In an example embodiment, the biosample may be a seed as described herein or a seed wash as described herein, for example, from an agricultural crop as described herein. When the biosample is a seed, the method 100 may include the steps of combining the seed with a solvent as described herein and agitating (e.g., shaking on a horizontal shaker, etc.) the seed and solvent together to recover spores, for example, from the seed and prepare the assay sample (e.g., at 104 in the method 100, etc.) In doing so, the solvent may include, for example, phosphate-buffered saline (PBS) and optionally, a surfactant as described herein, such as polyoxyethylene (20) sorbitan monooleate (commercially available as TWEEN® 80). It should be appreciated that the assay sample may include all or part of the biosample.

Additionally or alternatively, the biosample may be a liquid formulation as described herein. In some embodiments, the liquid formulation may include, or may be suspected of including bacteria, spores of bacteria, fungi, or a combination thereof. Whether a biosample is suspected of comprising bacteria, spores of bacteria, viruses, and/or fungi depends on whether the sample is believed to be sterile or to comprise microbes appropriate or suitable for promoting the growth of a plant and/or germination of a seed. In some instances, the biosample may be a liquid formulation intended to promote the growth of a plant and/or germination of a seed by providing growth- and/or germination-promoting bacteria and/or fungi. In other instances, the biosample may be a liquid formulation that is a general fertilizer intended to be sterile (e.g., a liquid formulation comprising no bacteria, spores of bacteria, viruses or fungi, etc.) In any case, when the biosample is a liquid formulation, the method 100 may include the steps of combining the liquid formulation with a solvent as described herein and again agitating (e.g., vortexing, etc.) the liquid formulation and solvent together to recover spores, if any, from the liquid formulation and prepare the assay sample (e.g., at 104 in the method 100, etc.) In doing so, again, the solvent may include, for example, PBS and optionally, a surfactant as described herein. It should be appreciated, again, that the assay sample may include all or part of the biosample.

The assay, then, may be performed directly on the assay sample or a portion thereof (e.g., at 106 in the method 100, etc.) In doing so, the assay may involve a polymerase chain reaction (PCR) assay, including, but not limited to, a quantitative PCR (qPCR) assay, a reverse transcription quantitative PCR (RT-qPCR) assay, a digital PCR (dPCR) assay, a viability PCR (vPCR) assay, etc. In a specific embodiment, the assay includes a qPCR assay or a dPCR assay.

In any embodiment, the assay (e.g., qPCR, dPCR, etc.) may be performed directly on the assay sample without isolating and/or purifying and/or concentrating and/or enriching the nucleotides (e.g., DNA or RNA, etc.) within the assay sample or biosample. Additionally or alternatively, the biosample may not be processed or may be minimally manipulated prior to preparing the assay sample. In such instances, the biosample may be not cultured nor are the cells within the biosample lysed or otherwise manipulated to isolate, extract, purify, and/or concentrate nucleotides (e.g., DNA or RNA, etc.) Instead, the biosample, or a portion or aliquot thereof, may be placed directly in an assay medium, or an assay medium may be added directly to the biosample, to prepare the assay sample (e.g., at 104 in the method 100, etc.) for analysis. That said, in some embodiments, the assay may be performed directly on the biosample or a portion thereof (e.g., without separate preparation of an assay sample, etc.)

In any embodiment, in connection with preparing the assay sample (e.g., at 104 in the method 100, etc.) and/or in connection with performing the assay (e.g., at 106 in the method 100, etc.), the assay sample or a portion thereof may be serially diluted and an assay may be performed directly on the serially diluted assay sample. This can be useful when the amount of bacteria and/or fungi present in the biosample is unknown and/or known to be sufficiently ample such that the Ct value is expected to be too low. Where an assay sample is diluted, determination of the equivalent CFUs may include a dilution modifier in the calculation to quantify (and/or quantitate) the bacterial and/or fungal strains present in the assay sample (e.g., at 112 in the method 100, etc.) Additionally, the dilution may be necessary to obtain a result from the assay to identify one or more bacterial and/or fungal strains present it the assay sample (e.g., at 110 in the method 100, etc.) The dilution modifier can be determined with any conventional technique known to the artisan. Additionally or alternatively, the assay may be performed in triplicate.

In any embodiment, the biosample and/or assay sample may be incubated with a dye, prior to performing the assay thereon or in connection therewith, that inhibits nucleotide amplification. In some embodiments, the dye may intercalate a nucleotide sequence. The dye may include, for example (and without limitation), Propidium Monoazide (PMA) or Ethidium Monoazide (EMA). In a further embodiment, the assay may utilize a dye that inhibits nucleotide amplification but does not include a probe sequence.

In connection with evaluating the results of the assay (e.g., at 108 in the method 100, etc.), the identifying presence of one or more bacterial and/or fungal strains may be based on establishing a background (or baseline) threshold value such that a result that is above the background value indicates that a particular bacterial strain and/or fungal strain is present in the assay sample, and thus the biosample (e.g., at 110 in the method 100, etc.) In instances where two or more bacterial and/or fungal strains are measured, the background value may be the same or different. Determination of the background value can be accomplished by accounting for the bacterial and/or fungal strain to be identified, the test performed, and the reagents used. For example, where the assay is qPCR, a cycle threshold (Ct) value may be selected, wherein if the fluorescence associated with the primer/probe pair (e.g., a combination, etc.) is above a certain Ct value, the bacterial and/or fungal strain associated with that combination is deemed to be identified and present.

In any embodiment, identification of the one or more bacterial and/or fungal strains as present in the assay sample (and, thus, the biosample) may be based on a fluorescent signal (e.g., a fluorescent signal from the probe sequence, etc.) detected in the assay sample and satisfying at least one background value (as generally discussed above).

Further, as part of evaluating the results of the assay, the methods herein may include preparation of a standard/reference curve to correlate results from the assay with an equivalent CFU for a particular bacterial and/or fungal strain, for example, to quantify the bacterial and/or fungal strain present in the assay sample, and thus the biosample (e.g., at 112 in the method 100, etc.) For example, a reference/standard curve may be generated by plotting Ct values for a microbial strain against log 10 values obtained from a CFU assay to determine if there is a linear response in the number of cycles needed to observe a fluorescent signal depending on the number of spores present in a PCR reaction. A linear response may indicate that the number of spores present in a sample may be estimated based on the number of PCR cycles that took for the signal to appear.

As a non-limiting example, a series of samples comprising known and varying CFUs of a particular bacteria (e.g., a serial dilution of the bacteria) may be prepared. And, qPCR may be performed on the series of bacterial samples and a Ct value for each dilution (representing a known CFU) may be determined. The Ct value of each dilution then may be correlated with the CFU of the bacteria or fungi in the dilution to establish a standard/reference curve which equates a given Ct value with an equivalent CFU. By this process, a determination may be made of whether a given bacterial and/or fungal strain is present in an assay sample (and thus in the corresponding biosample) (e.g., at 110 in the method 100, etc.), and a quantification (and/or quantitation) may be made of how much of the given bacterial and/or fungal strain is present in the assay sample (and again, the corresponding biosample) (e.g., at 112 in the method 100, etc.) (together with a description of an equivalent CFU value).

In any embodiment, the methods herein (including method 100, etc.) may utilize one or more combinations as described herein of a forward primer sequence, reverse primer sequence, and a probe sequence to achieve the operations described herein. The combination of the forward primer sequence, reverse primer sequence, and probe sequence may be specific for at least one bacterial and/or fungal strain, for example, at least one known bacterial and/or fungal strain. In any embodiment, the assay may utilize at least one of Combination 1, Combination 2, Combination 3, Combination 4, Combination 5, or Combination 6, all as described herein. In some embodiments, the assay sample may include at least one of a combination as described herein of a forward primer sequence, reverse primer sequence, and probe sequence, which are specific for at least one bacterial and/or fungal strain, for example, at least one known bacterial and/or fungal strain. In a further embodiment, the assay sample may comprise two or more combinations as described herein of a forward primer sequence, reverse primer sequence, and a probe sequence, which are specific for at least one bacterial and/or fungal strain, for example, at least one known bacterial and/or fungal strain. In this later embodiment, the two or more combinations may be selected from Combination 1, Combination 2, Combination 3, Combination 4, Combination 5, and Combination 6, as described herein.

In any embodiment, the methods described herein (including method 100, etc.) may be implemented to identify two or more bacterial and/or fungal strains. For example, the identification of one or more bacterial and/or fungal strains as present in the biosample may be based on a first fluorescent signal detected for the assay sample that satisfies a first background value. A second, different bacterial and/or fungal strain may also be identified as present in the biosample based on a second fluorescent signal detected for the assay sample that satisfies a second background value. In such instances, the first bacterial and/or fungal strain identified may be specific to a first of two or more combinations utilized in the method and the second bacterial and/or fungal strain may be specific to a second of the two or more combinations. The first background value may be the same or different as the second background value.

In connection therewith, the step of evaluating the results of the assay may include calculating a Ct value for each bacterial and/or fungal strain in the assay sample. For example, bacterial and/or fungal strains identified in the assay sample may be quantified by comparing a Ct value for each of the bacterial and/or fungal strains with a standard/reference curve for each of the bacterial and/or fungal strains and determining or quantifying an equivalent CFU. The standard/reference curve for each of the one or more bacterial and/or fungal strains may describe a correlation between the between the Ct value and the equivalent CFU value for each of the one or more of the bacterial and/or fungal strains.

Additionally or alternatively, in performing the assay on the assay sample (to identify two or more bacterial and/or fungal strains), the methods herein may utilize two or more of the combinations described herein, each comprising a forward primer sequence, a reverse primer sequence, and a probe sequence, wherein each combination is specific for a different bacterial and/or fungal strain. Identification of the one or more bacterial and/or fungal strains in the assay sample (and biosample) may be accomplished by differentially identifying at least two bacterial and/or fungal strains as present in the sample. For example, the assay may utilize a first combination and a second combination, where the first combination includes a first forward primer sequence, a first reverse primer sequence, and first probe sequence and where the second combination includes a second forward primer sequence, a second reverse primer sequence, and second probe sequence. The first combination and the second combination may each be specific to a different bacterial and/or fungal strain, whereby the different strains may both be identified by way of the methods herein.

Additionally, in implementing the methods herein to identify two or more bacterial and/or fungal strains, the methods may further comprise quantifying an equivalent CFU value for at least two bacterial and/or fungal strains present in the assay sample (and, thus, the biosample), which includes quantifying an equivalent CFU value for each of the at least two bacterial and/or fungal strains present in the sample.

Additionally or alternatively, any of the methods herein (including the method 100, etc.) may further include determining if one or more of the identified bacterial and/or fungal strains present in the assay sample (and, thus, the biosample) are viable based on the equivalent CFU value for each of the bacterial and/or fungal strains present in the sample satisfying one or more viability threshold values (e.g., particular to a given bacterial and/or fungal strain, generic to the strain(s), etc.) Thus, the methods herein may further include determining that the assay sample (and biosample) may be considered viable when the equivalent CFU satisfies a viability threshold value. As used herein, a “viability threshold value” is a value determined for a particular bacterial and/or fungal strain above which the bacterial and/or fungal strain will grow under appropriate conditions, or will be reasonably expected to grow under appropriate conditions. As used herein, “appropriate conditions” relate to a sufficient amount of nutrients, water, and sunlight for the bacterial and/or fungal strain to grow. In some embodiments, the viability threshold may be greater than about 1,000 CFUs or equivalent CFUs per gram of formulation or per individual seed. In other embodiments, the viability threshold may be greater about 10,000 CFUs or equivalent CFUs for the liquid formulation. Where appropriate, the viability threshold may further depend on a length of time that the biosample has been stored. For example, when the biosample has been stored for up to about three years prior to analysis, the viability threshold value may be greater than 1,000 CFUs or equivalent CFUs per seed or gram of formulation or greater than 10,000 CFUs or equivalent CFUs of the liquid formulation. In some instances, the viability threshold may be adjusted depending upon the conditions and length of storage of the biosample (e.g., the seed, seed wash, or liquid formulation, etc.), the type of seed or liquid formulation, etc.

In any embodiment, two biosamples (e.g., a first biosample and a second biosample, etc.) may be obtained from the same or different bulk supply at the same or different times. The method may include performing each step as described herein on both the first and the second biosample. In some circumstances, the first biosample may be obtained from the bulk supply at least one or more days (e.g., two days, three days, four days, five days, etc.) before the second biosample is obtained from the bulk supply. In some circumstances, the assay for the first biosample and the assay for the second biosample are performed at different times, such that at least one or more days (e.g., two days, three days, four days, five days, etc.) are present between the assays.

It is contemplated herein that any step of the methods described herein (e.g., method 100, etc.) can be performed in immediate sequential order or an interval of time may occur between any of the steps. For example, the steps of: preparing an assay sample from all or part of the biosample; performing an assay directly on the assay sample, or a portion of the assay sample; evaluating the results of the assay; identifying one or more bacterial and/or fungal strains as present in the biosample when the results are consistent with the presence of one or more bacterial and/or fungal strains; and quantifying an equivalent CFU value may be performed within a specified time period. The time period may include twelve hours or less, eight hours or less, six hours or less, four hours or less, and two hours or less.

Any method herein (including method 100) may further include applying a treatment to seed(s) (e.g., where the biosample includes the seed(s), etc.), including multiple bacteria and/or fungi, such that the multiple bacteria and/or fungi are disposed as a coating (or treatment) on the seed. In connection therewith, then, identifying one or more bacterial and/or fungal strain(s) as present in the biosample includes identifying the bacteria and/or fungi as present in the coating on the seed(s). In some examples, the multiple bacterial and/or fungal cells or spores applied to the seed(s) as the treatment may include about 2.5×10⁵ to about 3×10⁶ CFU of bacteria or fungi (or any other amount described herein) disposed as the coating per seed. In other examples, the treated seed may be stored for a period of time before it is used as a biosample, or the treated seed may be tested shortly after treatment and later after storage to determine stability of the microbial coating.

A further example method for identifying and/or quantitating one or more bacterial and/or fungal strains of a biosample is also provided herein. The biosample may include a seed as described herein or a liquid formulation as described herein configured as treatment for a seed. Here, the further method may include obtaining the biosample from a bulk supply of the seed or from a bulk supply of the liquid formulation, wherein the biosample includes multiple bacterial and/or fungal spores and one or more of a pesticide, an insecticide, and/or a fungicide; contacting (e.g., mixing, etc.) the biosample with a quantity of a solvent as described herein to prepare an assay sample; performing qPCR directly on the assay sample, or a portion thereof, without isolating and/or purifying DNA from the biosample; calculating a cycle threshold (Ct) value for the assay sample; identifying the bacterial and/or fungal spores as present in the biosample; and quantifying the bacterial and/or fungal spores present in the biosample as an equivalent colony-forming unit (CFU) value by comparing the Ct value with a standard/reference curve for said bacterial and/or fungal spores.

A further example method may include a multiplex assay. The multiplex assay may be used to detect and quantify more than one species simultaneously (e.g., in a non-sterile sample, a seed formulation containing more than one species, a soil sample containing more than one species, etc.) The method may use a different fluorescent label in the corresponding probes for each different species. Multiplexing may allow for an easy transition to higher throughput testing. For example, assays where one probe keeps the FAM label and the other is labeled with a dye that does not overlap its emission spectrum such as CY®3 or CY®5 may be performed. Also, primer specificity to a certain target species may allow for detection of spores of interest in mixed microbial samples.

Additionally, the qPCR assay described herein may be carried out in a well plate format (e.g., 96 well plate, 384 well plate, etc.) Further, the described methods may be automated to increase throughput and save resources.

It is contemplated that the methods described herein may be performed on a plant or seed, or biosample therefrom, or in a liquid formulation adapted to be applied to plants and/or seeds prior to, contemporaneously with, or subsequent to planting of said plant and/or seed.

### Deposit Information

A sample of the Bacillus velezensis strain referred to herein has been deposited with the Agricultural Research Service Culture Collection located at the National Center for Agricultural Utilization Research, Agricultural Research Service, U.S. Department of Agriculture (NRRL), 1815 North University Street, Peoria, Illinois 61604, U.S.A., under the Budapest Treaty on Feb. 14, 2019, and has been assigned the following accession number: NRRL B-67746.

A sample of the Bacillus amyloliquefaciens strain QST713 referred to herein has been deposited with the Agricultural Research Service Culture Collection located at the National Center for Agricultural Utilization Research, Agricultural Research Service, U.S. Department of Agriculture (NRRL), 1815 North University Street, Peoria, Illinois 61604, U.S.A., under the Budapest Treaty on Mar. 7, 1997, and has been assigned the following accession number: NRRL B-21661.

All strains described herein and having an accession number with the NRRL prefix have been deposited with the above-described respective depositary institution in accordance with the Budapest Treaty on the International Recognition of the Deposit of Microorganisms for the Purposes of Patent Procedure.

The following non-limiting examples are provided to further illustrate the present disclosure.

### Examples

### 1. Bacterial Strains

Bacillus velezensis (strain NRRL B-67746) is a gram-positive bacterial strain, was isolated from field trials for cotton in the United States.

Bacillus amyloliquefaciens (strain QST713) is a rod-shaped, Gram-positive, aerobic bacterium, which was first isolated from soil in the United States. It was initially classified as Bacillus subtilis QST713 (NRRL B-21661).

### 2. Seed Treatment

Spores were obtained by growing microbes in a liquid culture medium. Spores were frozen in 15% glycerol. The frozen cultures were thawed, diluted to a predetermined concentration with water and applied simultaneously with a proprietary formulation that includes fungicide, herbicide, antibacterial compounds, a polymer, colorant and water using a rotating mechanical drum at a rate which promoted an even distribution of the formulation and spores to achieve 10⁶ colony forming units (CFU) per seed. After these treatment steps, the treated seeds were tumbled for 2 minutes to allow the seeds to dry.

3. Recovery of Bacterial Spores on Seeds or from Liquid Formulation

Seed samples consisted of 30 seeds treated as described in the Seed Treatment above. To recover the spores from the seed samples, 30 mL of 1× Phosphate Saline Buffer containing 0.1% (w/v) TWEEN® 80 (PBS-T, pH 7.4) was added to the seeds in 50 ml conical tubes. They were placed on a horizontal shaker and agitated at 185 oscillations/min for 15 minutes.

The seed liquid formulation samples consisted of 1 mL proprietary formulation containing the spores mixed with 9 mL of 1× Phosphate Saline Buffer containing 0.1% (w/v) TWEEN® 80 (PBS-T, pH 7.4) in 50 mL conical tubes, which were vortexed for 10 seconds at high speed until the mixtures were uniform.

Recovered spores in suspension were used to create two dilution series, one in the same buffer and one in ultrapure water. The dilution series in buffer was used to perform a traditional CFU assay by plating aliquots onto TSA agar (15 g/L Pancreatic digest of casein, 5 g/L Papaic digest of soybean, 5 g/L sodium chloride, and 15 g/L agar) and incubating at 30° C. for 1 day. Colonies formed were counted for construction of the reference curve. The dilution series in water was used directly (i.e. no additional steps to extract or purify DNA) for quantitative PCR assays.

### 4. Primers and Probes

The primers and probes were designed using Integrated DNA Technologies' “Realtime PCR Tool” software, and were purchased from Integrated DNA Technologies (IDT) (Coralville, Iowa). Primers and probes were resuspended in a Tris-EDTA solution (Tris-EDTA 10/1, pH 8.0) to obtain a final concentration of 100 UM and stored at −20° ° C. These stocks were used to prepare working solutions at 10 μM by diluting 10× in Tris EDTA. The primer and probe sequences are presented in Table 2.

TABLE 2
Primer and Probe Sequences

| | Primer/ | Forward Primer | Reverse Primer | |
|---|---|---|---|---|
| Bacterial | Probe | Sequence | Sequence | Probe Sequence |
| Strain | Name | (5′ to 3′) | (5′ to 3′) | (5′ to 3′) |
| NRRL B- | 32-5 | CAATTACCTT | AAGCCATTTC | 6-FAM/AGAGGATAT/ZEN/ |
| 67746 | | TACCTGTCAC | TTGCATTCTT CC | GACACATGAAG AACTGCCA |
| | | AAGC | (SEQ ID NO: 2) | (SEQ ID NO: 3) |
| | | (SEQ ID NO: 1) | | |
| NRRL B- | 30-2 | GCAACAAGTC | GACTGGGTTT | 6-FAM/CCTCGATAA/ZEN/ |
| 67746 | | TTTCTCGTTC TG | CTAGCAGAGA AG | ACTTCTCTGTG CCATCAGG |
| | | (SEQ ID NO: 4) | (SEQ ID NO: 5) | (SEQ ID NO: 6) |
| NRRL B- | 30-3 | ACCTCGATAA | CTA AGA TGG | 6-FAM/TCATAGCCA/ZEN/ |
| 67746 | | ACTTCTCTGT GC | TTG ACT GGG TTT | TGTCCATCTTC TCTGCT |
| | | (SEQ ID NO: 7) | CT | (SEQ ID NO: 9) |
| | | | (SEQ ID NO: 8) | |
| NRRL B- | 85-2 | TGGTAAGCTT | CACTGGTAAG | 6-FAM/ACATCCTCC/ZEN/ |
| 67746 | | GGATCAGAA | TGATGCAGTG | GGTTCTCCTTCAGT AGT |
| | | (SEQ ID NO: 10) | AAA | (SEQ ID NO: 12) |
| | | | (SEQ ID NO: 11) | |
| NRRL B- | 86L-2 | CTGTATATCT | GTTAGAAACA | 6-FAM/TGGCTGAAG/ZEN/ |
| 67746 | | AAACCCTGGA | TGCAAGATGG | TCAAAGGGCCA ATTG |
| | | ATCTCTT | TCAG | (SEQ ID NO: 15) |
| | | (SEQ ID NO: 13) | (SEQ ID NO: 14) | |
| NRRL B- | Ser | GACGTATGGA | CCAAATTCCT | 6-FAM/TTCCCATTA/ZEN/ |
| 21661 | | TACACCTCTT | CAGAAGAGAG | ATATACTCAAT |
| | | TAAT | AG | TAGAGAACCT |
| | | (SEQ ID NO: 16) | (SEQ ID NO: 17) | (SEQ ID NO: 18) |

5. qPCR for Detection of Microbes

qPCR was performed in a final volume of 20 μL in a 96 PCR well plate using BioRad CFX1000 thermocycler (Bio-Rad Laboratories Inc, Hercules, California). Five μL of a dilution series sample, as described in the Recovery of Bacterial Spores on Seeds or from Liquid Formulation section above, was amplified with forward and reverse primers, and probe in IDT's PCR PRIMETIME® Gene Expression Master Mix (Cat No 1055772). Final concentrations of primers and probe were 250 nM and 125 nM, respectively, for both Bacillus velezensis strain (NRRL B-67746) and B. amyloliquefaciens strain QST713 (NRRL B-21661) (Table 3). PCR amplification conditions were: 95° C. for 3 min; 50 cycles of 15 seconds at 95ºC, followed by 58° C. for 1 min.

TABLE 3
Components and Concentrations of qPCR Reactions

| | Final Concentration |
|---|---|
| Component | in Reaction |
| 3.75 μL of MilliQ water | — |
| 10 μL of 2× of PCR reaction | 1× |
| mix (IDT) | |
| 0.5 μL of forward primer | 250 nM |
| 0.5 μL of reverse primer | 250 nM |
| 0.25 μL of FAM -labeled probe | 125 nM |
| 5 μL of corresponding dilution | — |
| series sample | |
| Total volume | 20 μL |

For each dilution tested, triplicate reactions were included. In addition, each 96 well plate included triplicate reactions where MilliQ water replaced the sample volume to serve as negative controls. Cycle threshold (Ct) was recorded for each reaction from a triplicate dilution set, averaged and used to create Log₁₀ CFU vs. Ct plot with the data from the on-plate assay. Results shown in the examples were obtained with primer and probe set “32-5” unless stated otherwise.

### 6. Quantification of Microbes by Plating

For quantification of Bacillus velezensis (NRRL B-67746) and the B. amyloliquefaciens QST713 (NRRL B-21661) cells/spores present on seeds or in liquid formulation, serial dilutions were made in Phosphate Saline Buffer containing 0.1% (w/v) tween 80 (PBS-T, pH 7.4) from the recovered samples. The recovered samples included two treatments which differ only in the date when the sample was treated. Each treatment was duplicated. The diluted samples were plated in triplicate on TSA agar culture media using a dilution volume that allowed for counting between 30-150 CFU on a plate after 16-24 hrs of incubation at 30° ° C. Once the colony count was obtained, CFU/seed was calculated, which was then converted to log 10 equivalent in order to compare the differences between samples. Any two or more samples were considered “the same” if the log₁₀ difference was less than 0.1. Accordingly, all four samples in Table 4 were considered equal regardless of two different treatments.

TABLE 4
CFU/Seed for Bacillus velezensis

| Sample | CFU/Seed | Log₁₀ | Log₁₀ Difference |
|---|---|---|---|
| Spore + Formulation from day 1 | 1.29 × 10⁶ | 6.11 | 0.00 |
| Spore + Formulation from day 1 | 1.16 × 10⁶ | 6.06 | 0.05 |
| Spore + Formulation from day 2 | 1.50 × 10⁶ | 6.18 | 0.00 |
| Spore + Formulation from day 2 | 1.43 × 10⁶ | 6.16 | −0.09 |

The obtained values of CFU/seed were further converted to CFU/mL for each one of the dilutions used in the PCR assay and the corresponding Log₁₀ values were calculated. Finally, the expected number of spores in the 5 μL to be used in a PCR reaction was calculated for assessing the detection limit of the PCR assay (Table 5).

TABLE 5
Conversion of CFU/seed to CFU/mL, log10 and CFU/5 μL

| Dilution | CFU/mL | Log₁₀ | CFU/5 μL |
|---|---|---|---|
| 1 (20×) | 135000 | 5.13 | 675 |
| 2 (2×) | 67500 | 4.83 | 338 |
| 3 (2×) | 33750 | 4.53 | 169 |
| 4 (2×) | 16875 | 4.23 | 84 |
| 5 (2×) | 8437 | 3.93 | 42 |
| 6 (2×) | 4218 | 3.63 | 21 |

### 7. Quantification of Microbes by PCR and Construction of Reference Curves

Similar serial dilutions were prepared for PCR assays as described in Example 6, except that after recovering the spores in PBS-T, the subsequent dilutions were prepared in MilliQ water, and the final sample dilution volume was adjusted to 1 mL. As described in the Quantification of Microbes by Plating section above, the PCR reactions were carried out in 20 μL reaction mixtures in triplicate in a 96 well plate using Biorad CFX1000. Five μL of the corresponding diluted spore samples were added in each reaction.

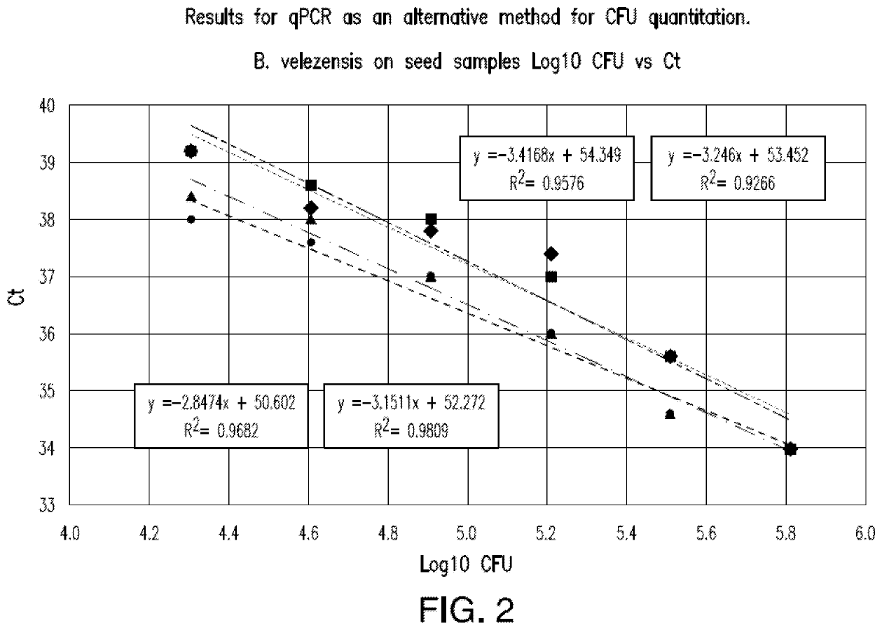

Ct values from PCR data for each serial dilution were obtained from the cycle vs fluorescent signal plot generated by the instrument. The Ct values were plotted against the log 10 CFU values obtained from the CFU on plate assay to determine if there was a linear response in the number of cycles needed to observe the fluorescent signal depending on the number of spores present in the PCR reaction. As shown in FIG. 2, the four samples showed a good linear correlation between the number of cycles needed to observe signal and the number of spores present in the reaction. The coefficient of determination (R²) ranged from 0.9266 to 0.9809. This indicates that the number of spores present in a sample can be estimated based on the number of PCR cycles that took for the signal to appear. In addition, it can be seen that the curves in the plot were separated in groups of two. The ones at the bottom corresponded to samples 3 and 4, while those at the top to samples 1 and 2. This result is expected based on the CFU numbers obtained from the plate assay. Even though any two or more samples were considered “the same” if the log₁₀ difference was less than 0.1, the PCR method is sensitive enough to pick up the small difference between the two groups of samples, where Samples 3 and 4 had slightly higher CFU values than Samples 1 and 2.

Once the reference curve was created, other samples were analyzed by PCR to obtain CFU values from the reference curve.

In order to validate that the method can be used with other types of samples and a different bacterial species, liquid formulation containing Bacillus amyloliquefaciens (NRRL B-21661) spores was used to create dilutions for both on plate count and PCR quantification. Four independent samples were withdrawn from the stock and used for the test.

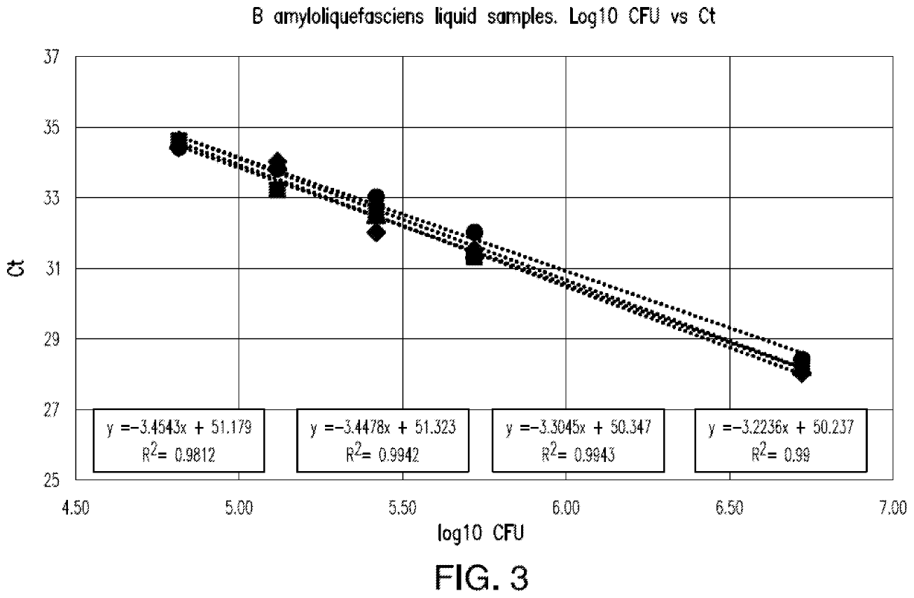

As shown in FIG. 3, the test with Bacillus amyloliquefaciens spores in liquid formulation also generated a linear response with a good fit. Thus, the method can be applied to different microbial species and types of samples.

### 8. Specificity of the PCR Assay

Since the primers and probes can be designed for species-specific DNA sequences, the PCR assays would allow for detection and quantification of only the target microbes in a sample. In order to test specificity, PCR reactions were set up using liquid samples with three different treatments: 1) spores of the species of interest (Bacillus velezensis) tested against the primers and probes for Bacillus amyloliquefaciens; 2) spores of B. amyloliquefaciens tested against the primers and probe for B velezensis; 3) spores from both species in the same sample where one of them was in excess over the other and tested against their corresponding primers and probes set. For the specificity test with single species, dilutions were made from liquid samples as described in the previous examples and used for the PCR assay.

To test specificity in a sample containing both Bacillus velezensis (NRRL B-67746) and B. amyloliquefaciens QST713 (NRRL B-21661), the spores were mixed at different ratios as shown in Table 6.

TABLE 6
Number of spores in 5 μL sample used for PCR and ratio
between the spores of the two species in the mixture

| B velezensis Spore# | B amyloliquefaciens Spore# | Ratio |
|---|---|---|
| 2.50 × 10⁶ | 1.30 × 10⁴ | 192:1 |
| 2.50 × 10⁶ | 1.30 × 10³ | 1923:1 |
| 2.50 × 10⁶ | 3.33 × 10² | 7519:1 |
| 5.40 × 10⁴ | 1.30 × 10⁵ | 1:2.4 |
| 1.30 × 10⁴ | 1.30 × 10⁵ | 1:10 |
| 6.50 × 10² | 1.30 × 10⁵ | 1:200 |

Both, the single spore samples and the mixtures were tested against primers 32-5 and Ser.

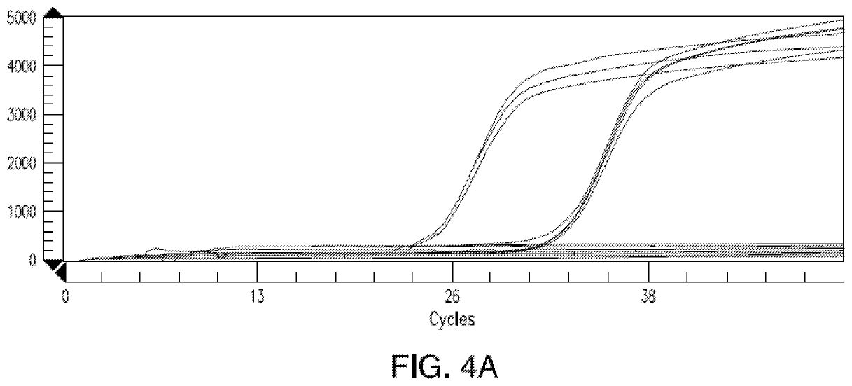

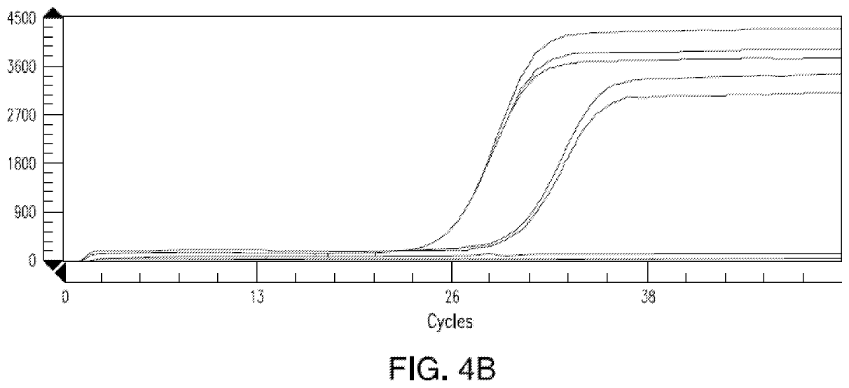

As shown in FIGS. 4A-4B, for both single spore samples, only those reactions containing the corresponding primer and probe set for the species being tested produced fluorescent signal. For all those reactions containing the primer and probe set for the other species, the fluorescent signal remained at the same level as the control samples without DNA.

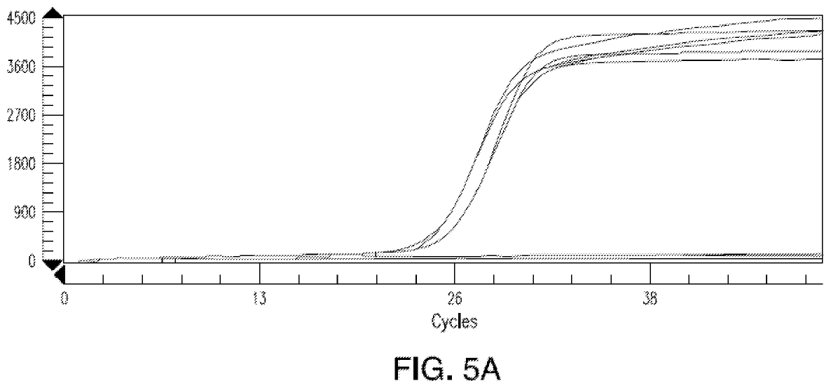

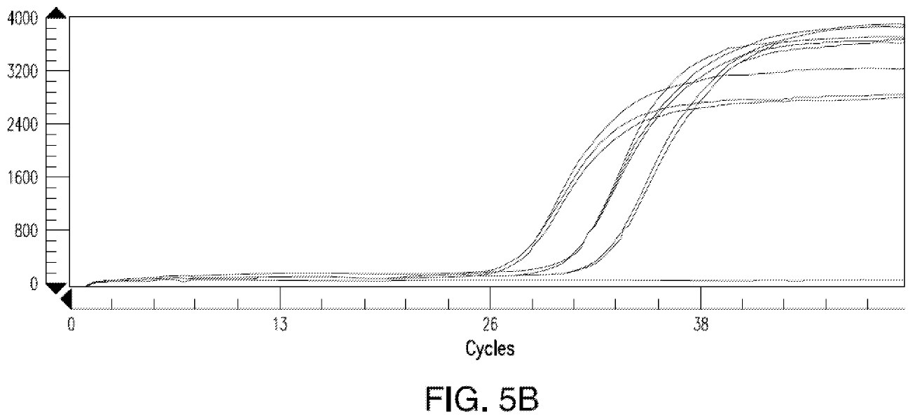

A similar result was obtained for the mixed samples containing one of the spores in excess over the other. FIGS. 5A-5B shows that in the mixture spore samples, the only signal generated corresponded to the target species even if the non-target spores were present at a concentration orders of magnitude higher.

Thus, the primers were specific for the target species and allowed for detection of the spores of interest in mixed microbial samples.

9. Quantification of B. velezensis with Different PCR Mixes

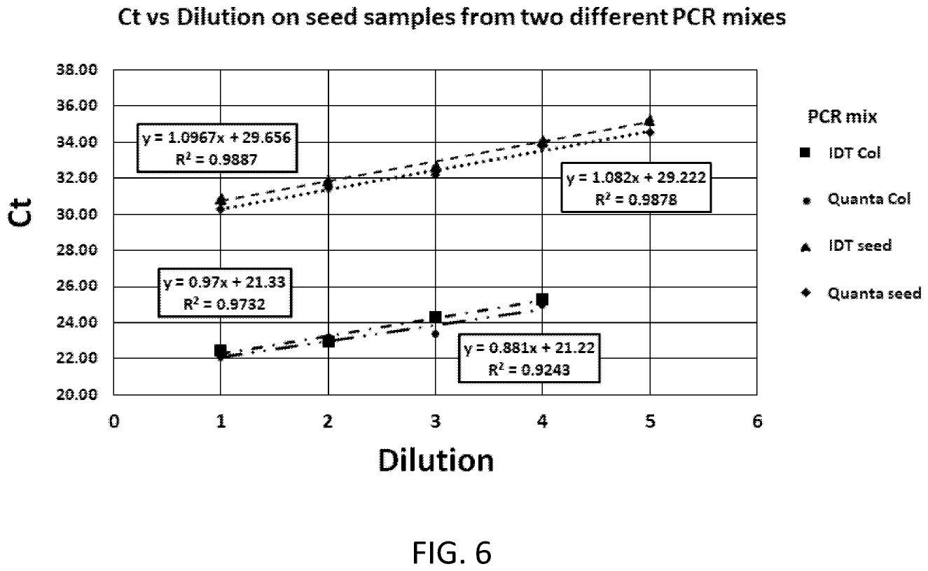

IDT's PCR PRIMETIME® Gene Expression Master Mix (Cat No 1055772) (referenced as IDT in FIG. 6) was compared to Quanta Bio's ULTRAPLEX™ 1-STEP THROUGHMIX® (4×) (Cat No 95166-100) (referenced as Quanta in FIG. 6). Their performance was evaluated with both on-seed samples (referenced as IDT seed and Quanta seed in FIG. 6) and colony plate samples (referenced as IDT Col and Quanta Col in FIG. 6) of B. velezensis.

Seed samples were processed and diluted as described above. For colony plate samples, one colony was picked, resuspended in 1 mL of water, and diluted as described above. The water volume was adjusted to reach the final 1× concentration for the Quanta Bio mix.

Reactions were performed with the same CFX1000 thermocycler and PCR amplification conditions as described above. Ct values were obtained and a dilution vs. Ct plot was created for the samples (FIG. 6).

Both qPCR mixes provided linear responses with on seed or colony samples. Colony samples, containing high concentrations of the microbe, generated signal in fewer cycles. Additionally, both mixes give similar Ct values for both samples tested.

10. qPCR Assay for On-Seed CFU Loads of B. Velezensis

Seed samples containing different CFU loads were prepared and analyzed according to the qPCR assay. CFU loads ranged from about 0.25× to about 3× of the normal application of B. velezensis (e.g., from a CFU load of about 2.50×10⁶ to a CFU load of about 3.0× 10⁶, etc. where the normal application or normal CFU load is about 1.0×10⁶; etc.)

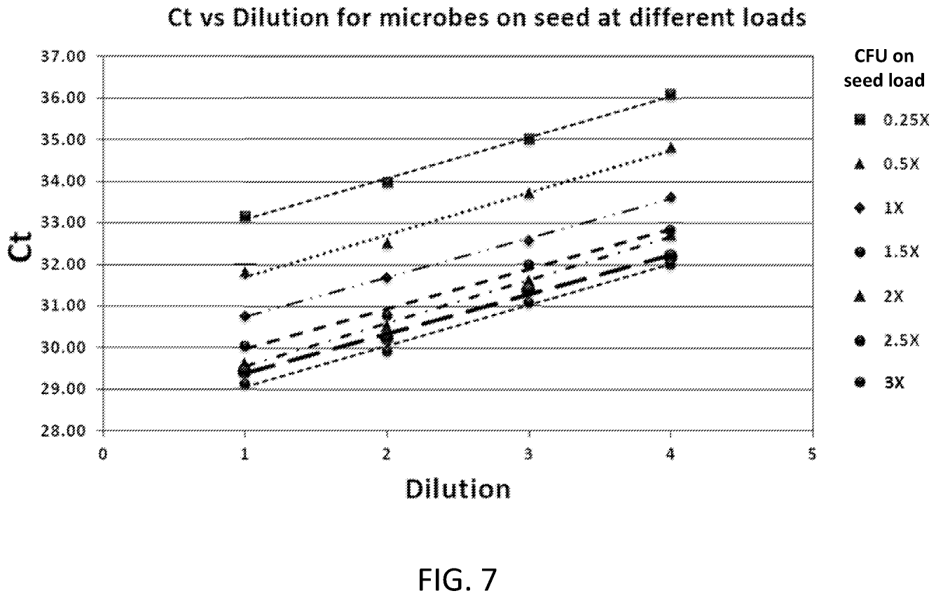

Samples were processed as above, and serial diluted (2×). qPCR reactions were performed as described above and Ct values for all samples and dilutions were obtained. A plot of dilution vs Ct was created for the samples (FIG. 7), in which the different loads are arranged in the plot from lowest (toward the top of the plot) to highest (toward the bottom of the plot). The plot shows a linear response for all samples.

Additionally, the number of cycles needed to generate signals and the position of the corresponding curve in the plot for each sample matches the expected results, i.e., lower microbial loads take longer time to generate signals and are above the “1×” line, while higher microbial loads generate signal faster and are below the “1×” line in the plot.

11. qPCR Assay in a 384-Well Plate Format

The assay performance herein was evaluated in a 384-well plate format.

Different seed samples (Samples 1-8) of B. velezensis were processed as described and dilutions series created. Reaction volumes were halved, without changing the final concentrations of the components. The qPCR assay was performed without changing cycle parameters and Ct values were obtained for all samples and dilutions.

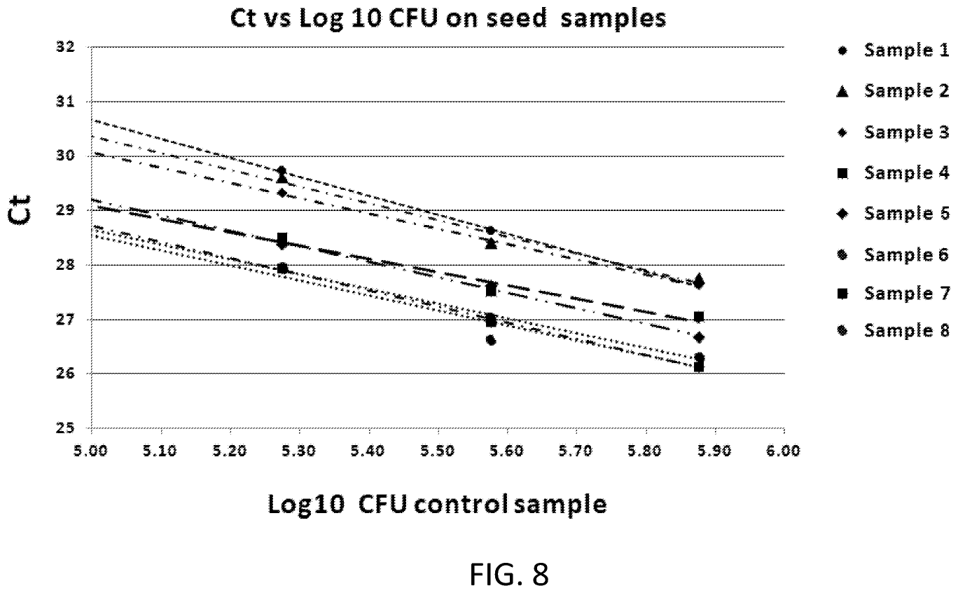

A Log 10 CFU control vs Ct plot was created (FIG. 8) and compared to values obtained in the 96 well format. The plot shows a linear response for all samples tested and the Ct values were similar to those obtained in the 96 well format.

### 12. Example Applications

In view of the above, it can be seen that the methods herein are able to specifically detect target species in a mixture, and it thus can be appreciated that the methods may further be adapted to a multiplex assay where more than one species is detected and quantitated simultaneously by using a different fluorescent label in the corresponding probes. Multiplexing allows an easy transition to higher throughput testing. For example, assays where one probe keeps the FAM label and the other is labeled with a dye that does not overlap its emission spectrum such as CY®3 or CY®5 may be performed.

The protocols described herein may also be applied to studies where a fast check on microbial numbers is needed, to determine possible loss of spores during seed treatment, and also to check microbial stability over time under different storage conditions. For example, a quality control (QC) application may be tested by preparing liquid formulations with different CFU titers and treating seed with them. Then the spores are recovered from both liquid and seed, and the CFU difference between them is evaluated to determine how many spores are lost during treatment. To test stability, spore numbers may be evaluated that remain on the treated sample by analyzing how many spores are detectable on the treated sample following storage for a period of time. By changing the storage conditions, the method determines which conditions affect bacterial spore counts over time.

Further, the methods herein may allow the quantitation of spores from samples regardless of the fact that they might be alive or dead. In other words, the methods quantitate total numbers of the target microorganisms. This application effectively provides a change from qPCR to viability PCR (vPCR) and an ability to determine numbers of live and dead microbes in a sample. The use of a DNA dye, propidium monoazide (PMA) allows quantitation of both live and dead spores.

In some implementations, spores may be recovered and split into two populations. One population of spores, then, may be treated with propidium monoazide (PMA) and the other not treated with PMA. Dilutions of both samples may be prepared for PCR and analyzed. In doing so, the PMA treated sample may generate fluorescent signal at later times compared to the untreated sample because the PMA should have penetrated the dead spores, bound to their DNA and made it unavailable for PCR amplification. Thus, by comparing the difference in number of cycles needed to generate signal between the treated and untreated sample, the method may be used to calculate how many spores were dead.

The methods herein may also be utilized with chips in digital PCR assays (dPCR), as well as droplet PCR assays, to perform droplet digital PCR (ddPCR). In connection therewith, switching from plate to chip or droplets may allow for more precise quantitation of CFU in a sample by splitting signals from the assay as (+) or (−) to be counted, and may also reduce an amount of sample needed for the assay as well as amounts of reagents.

The methods herein may also be utilized on or with substances other than plants, seeds, seed washes, and liquid formulations. For example, the methods may be utilized to identify and quantify (and/or quantitate) bacterial and/or fungal strains on one or more of plant tissues (e.g., from leaves, stalks, stems, flowers, roots, etc.) and washes thereof, plant biopsies and washes thereof, soil, fertilizer, etc. In such examples, the plant tissues, plant biopsies, soil, or fertilizer may comprise the biosample. Additionally and/or alternatively, the biosample may be an inside and/or interior portion of the plant, seed, plant tissue, or plant biopsy (e.g., a location other than the exterior of the plant, seed, plant tissue, or plant biopsy, etc.)

The foregoing description of the embodiments has been provided for purposes of illustration and description. It is not intended to be exhaustive or to limit the present disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the present disclosure, and all such modifications are intended to be included within the scope of the present disclosure.

Example embodiments have been provided so that this disclosure will be thorough, and will fully convey the scope to those who are skilled in the art. Numerous specific details are set forth such as examples of specific components, assemblies, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Throughout this disclosure, the numerical values represent approximate measures or limits to ranges to encompass minor deviations from the given values and embodiments having about the value mentioned as well as those having exactly the value mentioned. Other than in the working examples provided at the end of the detailed description, all numerical values of parameters (e.g., of quantities or conditions) in this specification, including the appended claims, are to be understood as being modified in all instances by the term “about” whether or not “about” actually appears before the numerical value. “About” indicates that the stated numerical value allows some slight imprecision (with some approach to exactness in the value; approximately or reasonably close to the value; nearly). If the imprecision provided by “about” is not otherwise understood in the art with this ordinary meaning, then “about” as used herein indicates at least variations that may arise from ordinary methods of measuring and using such parameters. For example, “about” may comprise a variation of less than or equal to 5%, optionally less than or equal to 4%, optionally less than or equal to 3%, optionally less than or equal to 2%, optionally less than or equal to 1%, optionally less than or equal to 0.5%, and in certain aspects, optionally less than or equal to 0.1%.

Specific values and/or specific material(s) disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9. In addition, disclosure of ranges includes disclosure of all values and further divided ranges within the entire range, including endpoints and sub-ranges given for the ranges.

The terminology used herein is for the purpose of describing particular example embodiments only and is not intended to be limiting. As used herein, the singular forms “a”, “an” and “the” may be intended to include the plural forms as well, unless the context clearly indicates otherwise. In other words, the articles “a” and “an” and “the” are used herein to refer to one or more than one (i.e., to at least one) of the grammatical object of the article. By way of example, “an element” means one or more elements. The terms “comprises,” “comprising,” “including,” and “having,” are inclusive and therefore specify the presence of stated features, integers, steps, operations, elements, components, and/or groups thereof, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

Although the terms first, second, third, etc. may be used herein to describe various elements, components, combinations, seeds, members and/or sections, these elements, components, seeds, members and/or sections should not be limited by these terms. These terms may be only used to distinguish one element, component, seed, member or section from another element, component, combination, seed, member or section. Terms such as “first,” “second,” and other numerical terms when used herein do not imply a sequence or order unless clearly indicated by the context. Thus, a first element, component, combination, seed, member or section discussed below could be termed a second element, component, combination, seed, member or section without departing from the teachings of the example embodiments.

Unless defined otherwise, all technical and scientific terms herein have the same meaning as commonly understood by one of ordinary skill in the art to which the present disclosure belongs. Although any methods and materials, similar or equivalent to those described herein, can be used in the practice or testing of the present disclosure, the non-limiting exemplary methods and materials are described herein.

What is claimed is:

1-32. (canceled)

33. A method for identifying and/or quantitating one or more bacterial and/or fungal strain(s) in a biosample, the method comprising:
(a) obtaining a biosample;
(b) preparing an assay sample comprising all or part of the biosample;
(c) performing an assay directly on the assay sample, or a portion thereof;
(d) evaluating the results of the assay;
(e) identifying one or more bacterial and/or fungal strain(s) as present in the biosample when the results are consistent with the presence of the one or more bacterial and/or fungal strain(s); and
(f) quantifying an equivalent colony-forming unit (CFU) value for the one or more bacterial and/or fungal strain(s) present in the biosample, as identified in step (e), by comparing the results of step (d) with a standard curve for the one or more bacterial and/or fungal strain(s).

34. The method of claim 33, wherein the assay performed directly on the assay sample is qPCR.

35. The method of claim 34, wherein performing the qPCR directly on the assay sample includes performing the qPCR on the assay sample without isolating and/or purifying DNA from the biosample.

36. The method of claim 35, wherein the qPCR utilizes one or more combination(s) of a forward primer sequence, a reverse primer sequence, and a probe sequence specific for at least one known bacterial and/or fungal strain.

37. The method of claim 36, wherein the one or more combination(s) of a forward primer sequence, a reverse primer sequence, and a probe sequence is selected from the group consisting of:
Combination 1 comprising SEQ ID NO:1, SEQ ID NO:2, and SEQ ID NO:3;
Combination 2 comprising SEQ ID NO:4, SEQ ID NO:5, and SEQ ID NO:6;
Combination 3 comprising SEQ ID NO:7, SEQ ID NO:8, and SEQ ID NO:9;
Combination 4 comprising SEQ ID NO:10, SEQ ID NO:11, and SEQ ID NO:12;
Combination 5 comprising SEQ ID NO:13, SEQ ID NO:14, and SEQ ID NO:15; and
Combination 6 comprising SEQ ID NO:16, SEQ ID NO:17, and SEQ ID NO:18.

38. (canceled)

39. The method of claim 34, wherein the qPCR utilizes two or more combinations of a forward primer sequence, a reverse primer sequence, and a probe sequence specific for at least one known bacterial and/or fungal strain.

40. The method of claim 39, wherein the two or more combinations are selected from the group consisting of:
Combination 1 comprising SEQ ID NO:1, SEQ ID NO:2, and SEQ ID NO:3;
Combination 2 comprising SEQ ID NO:4, SEQ ID NO:5, and SEQ ID NO:6;
Combination 3 comprising SEQ ID NO:7, SEQ ID NO:8, and SEQ ID NO:9;
Combination 4 comprising SEQ ID NO:10, SEQ ID NO:11, and SEQ ID NO:12;
Combination 5 comprising SEQ ID NO:13, SEQ ID NO:14, and SEQ ID NO:15; and
Combination 6 comprising SEQ ID NO:16, SEQ ID NO:17, and SEQ ID NO:18.

41-42. (canceled)

43. The method of claim 33, wherein step (d) comprises calculating a cycle threshold (Ct) value for the assay sample.

44. The method of claim 43, wherein step (f) comprises quantifying an equivalent CFU value for each of the one or more bacterial and/or fungal strain(s) present in the biosample as identified in step (e), by comparing the Ct value of step (d) with the standard curve for each of the one or more bacterial and/or fungal strain(s).

45. The method of claim 44, wherein the standard curve for each of the one or more bacterial and/or fungal strain(s) describes a correlation between the Ct value and the equivalent CFU value for each of the one or more bacterial and/or fungal strain(s).

46. The method of claim 33, wherein step (e) comprises identifying the one or more bacterial and/or fungal strain(s) as present in the biosample based on a fluorescent signal detected for the assay sample satisfying at least one background value.

47. The method of claim 34, wherein the qPCR is digital qPCR.

48. The method of claim 33, wherein the biosample is a seed or a seed wash.

49-52. (canceled)

53. The method of claim 33, wherein the biosample is a liquid formulation.

54-56. (canceled)

57. The method of claim 33, wherein a genus of the one or more bacterial strain(s) is selected from the group consisting of Bacillus, Bradyrhizobium, Paenibacillus, Pseudoacidovorax, Phytobacter, Pseudomonas, and Xanthomonas.

58. The method of claim 57, wherein the one or more bacterial strain(s) is selected from the group consisting of NRRL B-67746 and NRRL B-21661.

59-60. (canceled)

61. The method of claim 33, wherein the biosample includes one or more of a pesticide, an insecticide, and/or a fungicide.

62-69. (canceled)

70. The method of claim 33, further comprising incubating the biosample or assay sample with a dye that inhibits DNA amplification.

71. The method of claim 70, wherein the dye intercalates a nucleotide sequence.

72. The method of claim 70, wherein the dye is selected from the group consisting of fluorescein amidite (FAM), Aequorea victoria green fluorescent protein (VIC), 5-carboxytetramethylrhodamine (TAMRA), hexachloro-fluorescein (HEX), 2-[3-[1-[6-[(2,5-dioxo-1-pyrrolidinyl)oxy]-6-oxohexyl]-1,3-dihydro-3,3-dimethyl-5-sulfo-2H-indol-2-ylidene]-1-propen-1-yl]-1-ethyl-3,3-dimethyl-5-sulfo-3H-indolium (Cy3), 2-[5-[1-[6-[(2,5-dioxo-1-pyrrolidinyl)oxy]-6-oxohexyl]-1,3-dihydro-3,3-dimethyl-5-sulfo-2H-indol-2-ylidene]-1,3-pentadien-1-yl]-1-ethyl-3,3-dimethyl-5-sulfo-3H-indolium (Cy5), and 6-carboxy-4′,5′-dichloro-2′,7′-dimethoxyfluorescein succinimidyl ester (JOE).

73-79. (canceled)
